SEQUENCE LISTING (32 sequences)

SEQ ID NO: 1 - DNA, 2426 bp, Influenza B virus
agtactggtcgacctccgaagttgggggggagcagaagcggagcgttttcaagatgacattggccaaaattgaattgttaaaacaactgctaagggacaatgaagccaaaacagttttgaagcaaacaacagtagaccaatataacataataagaaaattcaatacatcaaggattgaaaagaatccttcactaaggatgaagtgggccatgtgttctaattttcccttggctctaaccaagggcgatatggcaaacagaatccccttggaatacaaagggatacaactcaaaacaaatgctgaagacataggaaccaaaggccaaatgtgctcaatagcagcagttacttggtggaatacatatggaccaataggagatactgaaggtttcgaaagggtctacgaaagcttttttctcagaaaaatgagacttgacaacgccacttggggccgaataacttttggcccagttgaaagagtgagaaaaagggtactgctaaaccctctcaccaaggaaatgcctccggatgaggcgagcaatgtgataatggaaatattgttccctaaagaagcaggaataccaagagaatccacttggatacatagggaactgataaaagaaaaaagagaaaaattgaaaggaacaatgataactccaatcgtactggcatacatgcttgaaagagaactggttgctcgaagaagattcttgccagtggcaggagcaacatcagctgagttcatagaaatgctacactgcttacaaggtgaaaattggagacaaatatatcacccaggagggaataaattaactgagtccaggtctcaatcaatgatagtagcttgtagaaaaataatcagaagatcaatagtcgcttcaaacccactggagctagctgtagaaattgcaaacaagactgtgatagatactgaacctttaaagtcatgtctggcagccatagacggaggtgatgtagcttgtgacataataagagctgcattaggactaaagatcagacaaagacaaagatttggacggcttgagctaaaaagaatatcaggaagaggattcaaaaatgatgaagaaatattaatagggaacggaacaatacagaagattggaatatgggacggggaagaggagttccatgtaagatgtggtgaatgcaggggaatactaaaaaagagtaaaatgaaactggaaaaactactgataaattcagccaaaaaggaggatatgagagatttaataatcttatgcatggtattttctcaagacactaggatgttccaaggggtgagaggagaaataaattttcttaatcgagcaggccaacttttatctccaatgtaccaactccaacgatattttttgaatagaagcaacgacctttttgatcaatgggggtatgaggaatcacccaaagcaagtgaactacatgggataaatgaatcaatgaatgcatctgactatacattgaaagggattgtagtgacaagaaatgtaattgacgactttagctctactgaaacagaaaaagtatccataacaaaaaatcttagtttaataaaaaggactggggaagtcataatgggagctaatgacgtgagtgaattagaatcacaagcacagctgatgataacatatgatacacctaaaatgtgggaaatgggaacaaccaaagaactggtgcaaaacacttatcaatgggtgctaaaaaacttggtgacactgaaggctcagtttcttctaggaaaagaggacatgttccaatgggatgcatttgaagcatttgagagcataattcctcagaagatggctggtcaatacagtggatttgcaagagcagtgctcaaacaaatgagagaccaggaggttatgaaaactgaccagttcataaagttgttgcctttttgtttctcaccaccaaaattaaggagcaatggggagccttatcaattcttaaaacttgtgttgaaaggaggaggggaaaatttcatcgaagtaaggaaagggtcccctctattttcctataatccacaaacagaagtcctaactatatgcggcagaatgatgtcattaaaagggaaaattgaagatgaagaaaggaatagatcaatggggaatgcagtattagcaggctttctcgttagtggcaaatatgacccagatcttggagatttcaaaactattgaagaacttgaaaagctgaaaccaggggaaaaggcaaacatcttactttatcaaggaaaaccagttaaagtagttaaaaggaaaaggtatagtgctttgtccaatgacatttcacaaggaattaagagacaaagaatgacagttgagtccatggggtgggccttgagctaatataaatttatccattaattcaatgaacgcaattgagtgaaaaatgctcgtgtttctact

SEQ ID NO: 2 - DNA, 2369 bp, Influenza B virus
agcagaagcggagcctttaagatgaatataaatccttattttctcttcatagatgtgcccgtacaggcagcaatttcaacaacattcccatacactggtgttcccccttattctcatggaacaggaacaggctacacaatagacaccgtgatcagaacgcatgagtactcaaacaaggggaaacagtacatttctgatgttacaggatgcacaatggtagatccaacaaatggaccattacccgaagataatgagccgagtgcctatgcgcaattagattgcgttttagaggctttggatagaatggatgaagaacacccaggtctttttcaagcagcctcacagaatgctatggaggccctaatggtcacaactgtagacaaattaacccaggggagacagacttttgattggacagtatgcagaaaccaacctgctgcaacggcactgaacacaacaataacctcttttaggttgaatgatttaaatggagccgacaaaggtggattaatacctttttgtcaggatatcattgattcattagaccgacctgaaatgactttcttctcagtaaagaatataaagaaaaaattgcctgccaaaaacagaaagggtttcctcataaagaggataccaatgaaggtaaaagacaaaataaccaaagtggaatacatcaaaagagcattatcattaaacacaatgacaaaagacgctgaaagaggcaaattgaaaagaagagcgattgccactgctggaatacaaatcagagggtttgtattagtagttgaaaacttggctaaaaatatatgtgaaaatctagaacaaagtggtttaccagtaggtggaaacgagaagaaagccaaactgtcaaacgcagtggccaaaatgctcagtaactgcccaccaggagggattagcatgacagtaacaggagacaatacaaaatggaatgaatgtttaaacccaagaatctttttggctatgactgaaagaataaccagagacagcccaatttggttcagggatttttgtagtatagcaccggtcctgttctccaataagatagcaagattggggaaagggtttatgataacaagcaaaacaaaaagactgaaggctcaaataccttgtcctgatctgtttagtataccattagaaagatataatgaagaaacaagggcaaaattgaaaaagctaaaaccattcttcaatgaagaaggaactgcatctttgtcgcctgggatgatgatgggaatgtttaatatgctatctaccgtgttgggagtagctgcactaggtatcaagaacattggaaacaaagaatacttatgggatggactgcaatcttctgatgattttgctctatttgttaatgcaaaggatgaagaaacatgtatggaaggaataaacgacttttaccgaacatgtaaattattgggaataaacatgagcaaaaagaaaagttactgtaatgagactggaatgtttgaatttacaagcatgttctacagagatggatttgtatctaattttgcaatggaactcccttcgtttggggttgctggagtaaatgaatcagcagatatggcaataggaatgacaataataaagaacaacatgatcaacaatgggatgggtccagcaacagcacaaacagccatacagttattcatagctgattatagatacacctacaaatgccacaggggagattccaaagtagaaggaaagagaatgaaaatcataaaggagttatgggaaaacactaaaggaagagatggtctattagtagcagatggtgggcccaacatttacaatttgagaaacttgcatatcccagaaatagtattaaagtataatctaatggaccctgaatacaaagggcggttacttcatcctcaaaatccctttgtgggacatttgtctattgaaggcatcaaagaggcagatataaccccagcacatggtccagtaaagaaaatggactacgatgcggtgtctggaactcatagttggagaaccaaaagaaacagatctatactaaacactgatcagaggaacatgattcttgaggaacaatgctacgctaaatgttgcaacctatttgaggcctgttttaacagtgcatcatacaggaagccagtgggtcaacatagcatgcttgaggctatggcccacagattaagaatggatgcacgattagattatgaatcagggagaatgtcaaaggatgattttgagaaagcaatggctcaccttggtgagattgggtacatataagcttcgaagatgtttatggggttattggtcaccattgaatacatgcgatacacaaatgattaaaatgaaaaaaggctcgtgtttctact

SEQ ID NO: 3 - DNA, 2305 bp, Influenza B virus
agcagaagcggtgcgtttgatttgtcataatggatacttttattacaagaaacttccagactacaataatacaaaaggccaaaaacacaatggcagaatttagtgaagatcctgaattgcaaccagcaatgctattcaatatctgcgtccatctagaggtttgctatgtaataagtgacatgaattttcttgacgaagaaggaaaagcacatatagcattagaaggacaagggaaagaacaaaacttgagaccacaatatgaagtaattgagggaatgccaagaaccatagcatggatggtccagagatccttagctcaagagcatggaatagagactcccaagtatctggctgatttgtttgattataaaaccaaaagatttatagaagttggaataacaaaaggattggctgatgattacttttggaaaaagaaagaaaagttgggaaatagcatggaactgatgatattcagctacaatcaagactactcgttaagtaatgaatcctcattggatgaggaagggaaagggagagtgctaagcagactcacagaacttcaggctgagttaagtctgaaaaacttatggcaagttctcataggagaggaagatgttgaaaagggaattgattttaaacttggacaaacaatatctagactaagggatatatctgttccggctggtttttccaattttgaaggaatgaggagctacatagacaatatagacccaaaaggagcaatagagagaaatctagcaaggatgtctcccttagtatcagtcacacctaaaaagttaacatgggaggacctaagaccgatagggcctcacatttatgaccatgagctaccagaagttccatataatgcctttcttctaatgtctgatgaactgggactggccaatatgactgagggaaaatccaaaaaaccgaagacattagccaaagaatgtctagaaaagtactcaacactacgggatcaaactgacccaatattaataatgaaaagcgaaaaagctaacgaaaatttcctatggaagctttggagagactgtgtaaacacaataagtaatgaggaaacaagtaacgagttacagaaaaccaattatgccaaatgggctacaggggacggattaacataccagaaaataatgaaagaagtagcaatagatgacgaaacaatgtgccaagaagagcctaaaatccctaacaaatgtagagtggctgcttgggttcaaacagagatgaatctattgagcactctgacaagtaaaagagctctggacctaccagaaatagggccagacatagcacccgtggagcatgtaggaagtgaaagaaggaaatactttgttaatgaaatcaactactgtaaggcctctacagttatgatgaagtatgtgctttttcacacttcattgttgaatgaaagcaatgccagcatgggaaaatacaaagtaataccaataaccaatagagtagtaaatgaaaaaggagaaagtttcgacatgctttacggtctagcggttaaaggacaatctcatctgaggggagatactgatgttgtaacagttgtaactttcgaatttagtggtacagatccaagagtggactcaggaaagtggccaaaatatactgtgtttaggattggctccctatttgtgagtgggagggaaaaatctgtgtacttgtattgccgagtgaatggcacaaataagatccaaatgaaatggggaatggaagctagaagatgtttgcttcaatcaatgcaacaaatggaggcaattgttgaacaggaatcatcaatacaaggatatgacatgaccaaagcctgtttcaagggagacagagtaaatagccccaaaactttcagtattggaactcaagaaggaaaactagtaaaaggatcctttggaaaaacactaagagtaatatttactaaatgcttgatgcactatgtatttggaaatgcccaattggaggggtttagtgccgagtctaggagacttctactgttgattcaagcattaaaggacagaaagggcccctgggtgttcgacttagagggaatgtattctggaatagaagaatgtattagcaacaacccttgggtaatacagagtgtatactggttcaatgaatggttgggctttgaaaaggaggggagtaaagtgttggaatcagtggatgaaataatggatgaataaaaggaaatggtactcaatttggtactattttgttcattatgtatctaaacatccaataaaaagaaccaagaatcaaaaatgcacgtgtttctact

SEQ ID NO: 4 - DNA, 1882 bp, Influenza B virus
agcagaagcagagcattttctaatatccacaaaatgaaggcaataattgtactactcatggtagtaacatccaatgcagatcgaatctgcactgggataacatcgtcaaactcaccacatgtcgtcaaaactgctactcaaggggaggtcaatgtgactggtgtaataccactgacaacaacacccaccaaatctcattttgcaaatctcaaaggaacagaaaccagagggaaactatgcccaaaatgccccaactgcacagatctggacgtagccttgggcagaccaaaatgcacgggaaaaataccctcggcaagagtttcaatactccatgaagtcagacctgttacatctgggtgctttcctataatgcacgacagaacaaaaattagacagctgcctaaccttctccgaggatacgaacatatcagattatcaactcataacgttatcaatgcagaaagtgcaccaggaggaccctacaaaattggaacctcagggtcttgccctaacgttaccaatggaaacggatttttcgcaacaatggcttgggccgtcccaaaaaacgacaaaaacaaaacagcaacagatccattaacaatagaagtaccatacatttgtacagaaggagaagaccaaattaccgtttgggggttccactctgataacgagatccaaatggcaaagctctatggggactcaaagccccagaagttcacctcatctgccaacggagtgaccacacattacgtttcacagattggtggcttcccaaatcaaacagaagacggaggactaccacaaagtggtagaattgttgttgattacatggtacaaaaatctgggaaaacaggaacaattacctatcaaagaggtattttattgcctcaaaaggtgtggtgcgcaagtggcaggagcaaggtaataaaaggatccttgcctttaattggagaagcagattgcctccacgaaaaatacggtggattaaacaaaagcaagccttactacacaggggaacatgcaaaggccataggaaattgcccaatatgggtgaagacacccttgaagctggccaatggaaccaaatatagacctcctgcaaaactattaaaggaaaggggtttcttcggagctattgctggtttcttagaaggaggatgggaaggaatgattgcaggttggcacggatacacgtcccatggggcacatggagtagcggtggcagcagaccttaagagcactcaagaggccataaacaagataacaaaaaatctaaactctttgagtgagctggaagtaaagaatcttcaaagactaagcggtgccatggatgaactccacaacgaaatactagaactagacgagaaagtagatgatctcagagctgatacaataagctcacaaatagaactcgcagtcctgctttccaatgaaggaataataaacagtgaagatgaacatctcttggcgcttgaaagaaagctgaagaaaatgctggggccctctgctgtagagatagggaatggatgctttgaaaccaaacacaagtgcaaccagacctgtctcgacagaatagctgctggtacctttgacgcaggagaattttctctccccacctttgattcactgaatattactgctgcatctttaaatgacgatggattggataatcataccatactgctttactactcaactgctgcctccagtttggctgtaacactgatgatagctatctttgttgtttatatggtctccagagacaatgtttcttgctccatctgtctataagggaagttaagccctgtgttttcctttgttgtagtgcttgtttgcttgttgccattacaaagaaacgttattgaaaaatgctcttgttact

SEQ ID NO: 5 - DNA, 1844 bp, Influenza B virus
agcagaagcacagcattttcttgtgaacttcaagcaccagtaaaagaactgaaaatcaaaatgtccaacatggatattgacggtataaacactgggacagttgacaaaacaccggaagaaataacttctggaaccagtgggacaaccagaccaatcattagaccagcaacccttgccccactaagcaacaaacgaacccgtaacccatccccggaaagagcaactacaagcagtgaagatgatgtcggaaggaaaacccaaaagaaacagaccccgacagagataaagaagagcgtctacaacatggtggtgaaactgggcgaattctataaccagatgatggtcaaagctggactcaatgatgacatggagagaaacctaatccaaaatgcgcatgccgtggaaagaattctattggctgccactgatgacaagaaaaccgatttccagaagaaaaagaatgccagagatgtcaaagaagggaaagaagaaatagatcacaacaaaacaggaggcaccttttataagatggtaagagatgataaaaccatctactttagccctataagaattacctttttaaaagaagaggtgaaaacaatgtacaaaaccaccatggggagtgatggcttcagtggactaaatcacataatgattgggcattcacagatgaatgatgtctgtttccaaagatcaaaggcactaaaaagagttggacttgatccttcattaatcagtacctttgcgggaagcacagtccccagaagatcaggtgcgactggtgttgcaatcaaaggaggtggaactttagtggctgaagccattcgatttataggaagagcaatggcagacagagggctattgagagatatcaaagccaagactgcctatgaaaagattcttctgaatctaaaaaacaaatgctctgcgcctcaacaaaaggctctagttgatcaagtgatcggaagcagaaatccggggattgcagacattgaagatctaaccctgcttgctcgtagtatggtcgttgttaggccctctgtggcaagcaaagtggtgcttcccataagcatttacgccaaaatacctcaactagggttcaatgttgaagagtactctatggttgggtacgaagccatggctctttacaatatggcaacacctgtgtccatattaagaatgggagatgatgcaaaagataaatcgcaattattcttcatgtcttgcttcggagctgcctatgaagacctgagagttttgtctgcattgacaggcacagaattcaaacctagatcagcattaaaatgcaagggtttccatgttccagcaaaggaacaagtagaaggaatgggagcagctctgatgtccatcaagctccagttttgggctccaatgaccagatctggggggaacgaagtaggtggagacggagggtctggccaaataagctgcagcccagtgtttgcagtggaaagacctattgctctaagcaagcaagctgtaaggagaatgctatcaatgaatattgagggacgtgatgcagatgtcaaaggaaatctactcaagatgatgaatgactcaatggctaagaaaaccagtggaaatgctttcattgggaagaaaatgtttcaaatatcagacaaaaacaaaaccaatcccattgaaattccaattaagcagaccatccccaatttcttctttgggagggacacagcagaggattatgatgacctcgattattaaagcaacaaaatagacactatgactgtgattgtttcaatacgtttggaatgtgggtgtttattcttattaaaataaatataaaaaatgctgttgtttctact

SEQ ID NO: 6 - DNA, 1557 bp, Influenza B virus
agcagaagcagagcatcttctcaaaattgaagcaaataggccgaaaatgaacaatgctaccctcaactatacaaacgttaaccctatttctcacatcagggggagtattattatcactatatgtgtcagcttcactgtcatacttactatattcggatatattgctaaaattcccatcaacagaaattactgcaccaacaatgccattagattgtgcaaacgcatcaaatgttcaggctgtgaaccgttctgcaacaaaaggggtgacacttcttctcccagaaccggagtggacatacccgcgtttatcttgcccgggctcaacctttcagaaagcactcctaattagccctcatagattcggagagaccaaaggaaactcagctcccttgataataagggaaccttttattgcttgtggaccaaaggaatgcaaacactttgctctaacccactatgcagcccaaccagggggatactacaatggaacaagaggagacagaaacaagctgaggcatctaatttcagtcaaattgggcaaaatcccaacagtagaaaactccattttccacatggcagcatggagcgggtccgcatgtcatgatggtaaggaatggacatatatcggagttgatggccctgacaataatgcattgctcaaaataaaatatggggaagcatatactgacacataccattcctatgcaaacaacatcctaagaacacaagaaagtgcctgcaattgcatcggaggaaattgttatcttatgataactgatggctcagcttcaggtgttagtgaatgcagatttcttaaaattcgagagggccgaataataaaagaaatatttccaacaggaagaatacaacatactgaagaatgcacatgcggatttgctagcaataaaaccatagaatgtgcctgtagagataacagttacacagcaaaaagaccctttgtcaaattaaacgtggagactgatacagcagaaataagattgatgtgcacaaagacttatttggacacccccagaccagaggatggaagcataactgggccttgtgaatctaatggaggcaaagggagtggaggcatcaagggaggatttgtccatcaaagaatggcatccaagattggaaggtggtactctcgaacgatgtctaaaactgaaaggaaggggatggggctgtatgtcaagtataatggagacccatgggctgacagtgatgcccttgtttttagtggagtaatggtttcaatggaagaacctggttggtactcctttggcttcgaaataaaagacaagaaatgtgatgtcccctgtattgggatagagatggtacatgatggtggaaaagagacttggcactcagcagctacagccatttactgtttaatgggctcaggacagctgctgtgggacactgccacaggtgttaatatgactctgtaatggaggaatggttgagtctgctctaaaccctttgttcctattttgtttgaacaattgtccttactaaacttaattgtttctgaaaaatgctcttgttactact

SEQ ID NO: 7 - DNA, 1190 bp, Influenza B virus
agcagaagcaggcactttcttaaaatgtcgctgtttggagacacaattgcctacttgctttcattgacagaagatggagaaggcaaaccagaactagcagaaaaattacactgttggtttggtgggaaagaatttgacctagactctgccttagaatggataaaaaacaaaagatgcttaactgatatacaaaaagcactaattggtgcctctatatgctttttaaaacccaaagaccaggaaagaaaaagaagattcatcacagagcccttatcaggagtgggaacaacagcaacaaaaaagaaaggcctgattctggctgagagaaaaatgagaagatgtgtgagctttcatgaagcatttgaaatagcagaaggccatgaaagctcagcgctactatactgtctcatggtcatgtacctgaatcctggaaattattcaatgcaagtaaaactaggaacgctctgtgctttgtgcgagaaacaagcatcacattcacacagagctcatagcagagcagcgagatcttcagtgcctggagtgagacgagaaatgcagatggtctcagctatgaacacagcaaaaacaatgaatggaatgggaaaaggagaagacgtccaaaagctggcagaagagctgcaaagcaacattggagtgctgagatctcttggggcaagtcaaaagaatggggaaggaattgcaaaggatgtaatggaagtgctaaagcagagctctatgggaaattcagctcttgtgaagaaatatctataatgctcgaaccatttcagattctttcaatttgttcttttatcttatcagctctccatttcatggcttggacaatagggcatttgaatcaaataaaaagaggaataaacatgaaaatacgaataaaaagtccaaacaaagagacaataaacagagaggtatcaattttgagacacagttaccaaaaagaaatccaggccaaagaaacaatgaaggaagtactctctgacaacatggaggtattgagtgaccacataataattgaggggctttctgccgaagaaataataaaaatgggtgaaacagttttggagatagaagaattgcattaaattcaatttttgctgtatttcttactatgcatttaagcaaattgtaatcaatgtcagcaaataaactggaaaaagtgcgttgtttctact

SEQ ID NO: 8 - DNA, 1097 bp, Influenza B virus
agcagaagcagaggatttgtttagtcactggcaaacaggaaaaatggcgaacaacatgaccacaacacaaattgaggtgggtccgggagcaaccaatgccaccataaactttgaagcaggaattctggagtgctatgaaaggctttcatggcaaagagcccttgactaccctggtcaagaccgcctaaacagactaaagagaaaattagagtcaagaataaagactcacaacaaaagtgagcctgaaagtaaaaggatgtcccttgaagagagaaaagcaattggagtaaaaatgatgaaagtactcctatttatggatccgtctgctggaattgaagggtttgagccatactgtatgaaaagttcctcaaatagcaactgtacgaaatacaattggaccgattacccttcaacaccagggagatgccttgatgacatagaagaagaaccagaggatgttgatggccccactgaaatagtattaagggacatgaacaacaaagatgcgaggcaaaagataaaggaggaagtaaacactcagaaagaagggaagttccgtttgacaataaaaagggatatgcgtaatgtattgtccttgagagtgttggtaaacggaacattcctcaaacatcccaatggatacaagtccttatcaactctgcatagattgaatgtatatgaccagagtggaaggcttgttgctaaacttgttgctactgatgatcttacagtggaggatgaagaagatggccatcggatcctcaactcactcttcgagcgtcttaatgaaggacattcaaagccaattcgagcagctgaaactgcggtgggagtcttatcccaatttggtcaagagcaccgattatcaccagaagagggagacaattagactggtcacggaagaactttatcttttaagtaaaagaattgatgataacatattgttccacaaaacagtaatagctaacagctccataatagctgacatggttgtatcattatcattattagaaacattgtatgaaatgaaggatgtggttgaagtgtacagcaggcagtgcttgtgaatttaaaataaaaatcctcttgttactact

SEQ ID NO: 9 - DNA, 29 bp, Artificial Sequence
  Primer
ccgaagttgggggggagcagaagcggagc

SEQ ID NO: 10 - DNA, 30 bp, Artificial Sequence
  Primer
ggccgccgggttattagtagaaacacgagc

SEQ ID NO: 11 - DNA, 29 bp, Artificial Sequence
  Primer
ccgaagttgggggggagcagaagcggagc

SEQ ID NO: 12 - DNA, 30 bp, Artificial Sequence; identical to SEQ ID NO: 10
  Primer

SEQ ID NO: 13 - DNA, 29 bp, Artificial Sequence
  Primer
ccgaagttgggggggagcagaagcggtgc

SEQ ID NO: 14 - DNA, 30 bp, Artificial Sequence
  Primer
ggccgccgggttattagtagaaacacgtgc

SEQ ID NO: 15 - DNA, 29 bp, Artificial Sequence
  Primer
ccgaagttgggggggagcagaagcagagc

SEQ ID NO: 16 - DNA, 30 bp, Artificial Sequence
  Primer
ggccgccgggttattagtagtaacaagagc

SEQ ID NO: 17 - DNA, 29 bp, Artificial Sequence
  Primer
ccgaagttgggggggagcagaagcagagc

SEQ ID NO: 18 - DNA, 30 bp, Artificial Sequence; identical to SEQ ID NO: 16
  Primer

SEQ ID NO: 19 - DNA, 29 bp, Artificial Sequence
  Primer
ccgaagttgggggggagcagaagcacagc

SEQ ID NO: 20 - DNA, 30 bp, Artificial Sequence
  Primer
ggccgccgggttattagtagaaacaacagc

SEQ ID NO: 21 - DNA, 32 bp, Artificial Sequence
  Primer
ccgaagttgggggggagcagaagcacgcactt

SEQ ID NO: 22 - DNA, 33 bp, Artificial Sequence
  Primer
ggccgccgggttattagtagaaacaacgcactt

SEQ ID NO: 23 - DNA, 32 bp, Artificial Sequence
  Primer
ccgaagttgggggggagcagaagcagaggatt

SEQ ID NO: 24 - DNA, 33 bp, Artificial Sequence
  Primer
ggccgccgggttattagtagtaacaagaggatt

SEQ ID NO: 25 - DNA, 22 bp, Artificial Sequence
  Primer
ctcaatttgtgttgtggtcatg

SEQ ID NO: 26 - DNA, 32 bp, Artificial Sequence
  Primer
tggaggatgaagaagatggccatcggatcctc

SEQ ID NO: 27 - protein, 286 aa, Unknown
  Receptor Binding Domain of the MERS coronavirus
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVI

SEQ ID NO: 28 - protein, 238 aa, Unknown
  Receptor Binding Domain of SARS-CoV-2
FTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNF

SEQ ID NO: 29 - DNA, 20 bp, Artificial Sequence
  Primer
gacatacttatgaggatgtc

SEQ ID NO: 30 - DNA, 22 bp, Artificial Sequence
  Primer
ctgaaagcttgacatggtgttg

SEQ ID NO: 31 - DNA, 26 bp, Artificial Sequence
  Primer
gacatactgtgaggatgtcaaaaatg

SEQ ID NO: 32 - DNA, 24 bp, Artificial Sequence
  Primer
ctgaaagcttgacacagtgtttgg

(19) United States
(12) Patent Application Publication
(10) Pub. No.: US 2024/0050558 A1
(43) Pub. Date: Feb. 15, 2024

(54) COMPOSITIONS IMMUNOGENIC AGAINST INFLUENZA AND SARS CORONAVIRUS 2, METHODS OF MAKING AND USING THEREOF

(71) Applicant: Versitech Limited, Hong Kong (CN)

(72) Inventors: Honglin Chen, Hong Kong (CN); Pui Wang, Hong Kong (CN); Zhiwei Chen, Hong Kong (CN); Kwok-Yung Yuen, Hong Kong (CN)

(21) Appl. No.: 18/267,931

(22) PCT Filed: Dec. 10, 2021
(86) PCT No.: PCT/CN2021/137112

§ 371 Date: Jun. 16, 2023

Related U.S. Application Data
(60) Provisional application No. 63/127,062, filed on Dec. 17, 2020.
(60) Provisional application No. 63/131,121, filed on Dec. 28, 2020.

(51) Int. Cl. A61K 39/215; A61P 31/14; C07K 14/165; A61K 9/00

(52) U.S. Cl. CPC A61K 39/215; A61P 31/14; C07K 14/165; A61K 9/0043; A61K 2039/5254

(57) ABSTRACT

Live attenuated viruses for protection against the novel coronavirus Sars-CoV-2 are provided. The live attenuated chimeric virus strains are based on a live attenuated influenza A or B virus (LAIVA/B), used a master backbone, which includes deletion of the viral virulence element, the NS1 (non-structural protein 1) (DeLNS1), engineered to express one or more antigens of the Sars-CoV-2 (herein, CoV2Ag). The chimeric virus strain is referred to generally herein, as DelNS1-A/B-Sars-CoV-2-CoV2Ag. The DelNS1-A/B-Sars-CoV-2-CoV2Ag strain preferably shows spontaneous cold adaption with preference to grow at 30-33° C. Compositions including the chimeric virus also provided as a co-composition with a LAIVA/B. The DelNS1-A/B-Sars-CoV-2-CoV2Ag strain can be used to protect a subject in need thereof, against a challenge of Sars-CoV-2. The co-compositions can be used to protect a subject in need thereof, against a challenge of Sars-CoV-2 and influenza A and/or B.

This international patent application claims the benefits of U.S. Provisional Patent Application No. 63/127,062 filed on Dec. 17, 2020 and U.S. Provisional Patent Application No. 63/131,121 filed on Dec. 28, 2020, the entire contents of which are incorporated by reference for all purpose.

### FIELD OF THE INVENTION

This invention is generally in the field of immunogenic compositions for eliciting an immune response against a Sars-CoV-2 infection, or Sars-CoV-2 and influenza infections.

### BACKGROUND OF THE INVENTION

The novel coronavirus (Severe Acute Respiratory Syndrome Coronavirus 2, or Sars-CoV-2) which emerged in 2019, remains a pressing public health crisis. There are two possibilities of the subsequent prevalence: (1) Sars-CoV-2 will disappear from humans after a huge intervention measures currently implanted by many countries; (2) Sars-CoV-2 may become a common cold virus and continue to circulate in humans, like other human coronavirus. There are three coronaviruses that have crossed species barriers and infected human since 2002/2003 of SARS coronavirus. It is reasonably to believe that other coronavirus from animal sources may emerge and infect humans in future. A rapid responsive and effective vaccine is needed for the current ongoing Sars-CoV-2 pandemic and future emerging coronavirus. Further, Humans do not have preexisting immunity to Sars-CoV-2 which has resulted in a pandemic leading to significant mobility and mortality worldwide. A vaccine for prevention of infection by Sars-CoV-2 is urgently needed. Combination vaccines for prevention of Sars-CoV-2 and influenza virus, are also needed.

Novel strategies to develop effective vaccines against the Sars-CoV-2 with properties to provide broad cross protective activity, and which can be co-administered with influenza virus immunogenic compositions are necessary. Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

It is an object of the present invention to provide a safe and effective live attenuated coronavirus.

It is also an object of the present invention to provide methods of generating live attenuated coronavirus vaccine.

It is also an object of the present invention to provide compositions immunogenic against SARS-2-CoV-2 and the influenza virus.

It is a further object of the present invention to provide methods of eliciting an immune response against coronavirus in a mammal.

It is a further object of the present invention to provide methods of eliciting an immune response against coronavirus and influenza virus in a mammal.

Compositions immunogenic against SARS-CoV-2, and co-compositions immunogenic against SARS-CoV-2 and the influenza virus and methods of using the same, are disclosed. The SARS-CoV-2 immunogenic compositions in include chimeric viruses made from a genetically modified influenza virus expressing antigens for the influenza virus and SARS-CoV-2. The co-compositions include the chimeric virus in combination with a live attenuated influenza virus A or B (LAIVA/B), which is attenuated in view of the modification to delete viral virulence element, the NS1 (non-structural protein 1) (DeLNS1).

The chimeric viruses are built on the backbone of LAIVA/B. The chimeric virus strain resulting from DelNS1 live attenuated influenza virus A or B (LAIVA/B), and expressing a SARS-CoV-2 antigen (CoV2Ag) is referred to generally herein, as A/B-Strain-DelNS1-Sars-CoV-2-CoV2Ag, with specific chimeric virus differing in name, depending on the CoV-2Ag being expressed and on the influenza virus backbone i.e., “A-Strain” or “B-Strain” refers to the specific influenza A or B virus strain. The chimeric virus strain resulting from DelNS1 LAIVA, and expressing a CoV2Ag is referred to generally herein, as -A-Strain-DelNS1-Sars-CoV-2-CoV2Ag. The chimeric virus strain resulting from DelNS1 LAIVB, and expressing a CoV2Ag is referred to generally herein, as B-Strain-DelNS1-Sars-CoV-2-CoV2Ag. The preferred CoV2Ag is the Sars-CoV-2 RBD (receptor binding domain). Thus, a chimeric virus expressing the Sars-CoV-2 RBD, is referred to generally as A/B-Strain-DelNS1-Sars-CoV-2-RBD.

Preferred chimeric vaccine strains based on an influenza A backbone include CA04-DelNS1-Sars-CoV-2-RBD ; HK68-DelNS1-Sars-CoV-2-RBD; 4801-DelNS1-Sars-CoV-2-RBD and H1N1(2019)-DelNS1-Sars-CoV-2-RBD strains. In one embodiment, a preferred LAIV backbone is a passage adapted strain A/California/04/2009 (a/ca/04/2009; CA04) virus, which includes deletion of the viral virulence element, the NS1 (non-structural protein 1), herein, CA04-DelNS1, and preferably includes two adaptive mutations located in the NP (D101N) and NEP (E95G) genes.

A preferred LAIVB backbone is DelNS1-B8038 (ATCC Deposit No. PTA-125209), and referred to herein interchangeably with DelNS1-8038B. A particularly preferred chimeric virus strains based on influenza B backbone is B8038-DelNS1-Sars-CoV-2-RBD.

Immunogenic compositions including a mixture of virus using influenza-based virus expressing SARS-CoV-2 antigen and influenza-based virus expressing various hemagglutinin (HA) and neuraminidase (NA) of influenza, herein after, co-compositions are disclosed. In these embodiments, the immunogenic compositions include a mixture of an influenza-based virus expressing a SARS-CoV-2 antigen as disclosed herein, and an influenza-based virus expressing various hemaglutinin (HA) and neuraminidase (NA) of influenza. The disclosed mixture is immunogenic against the influenza virus and the SARS-CoV-2.

In one embodiment, the co-compositions include A/B-Strain-DelNS1-Sars-CoV-2-CoV2Ag and a Live attenuated influenza virus (LAIV) A (“LAIVA”) and/or a Live attenuated influenza virus (LAIV) B (“LAIVB”), and in optional embodiments, wherein the LAIV A/B is engineered to express various hemaglutinin (HA) and neuraminidase (NA) of influenza. The LAIVA and LAIVB are attenuated virus, in view of the modification to delete viral virulence element, the NS1 (non-structural protein 1) (DeLNS1). The LAIVB from Victoria lineage is preferred, with a deletion of the viral virulence element, the NS1 (non-structural protein 1). The LAIVB additionally includes adaptive nucleotide mutations (AM) in segments 3 and 5-7 as follows: PA(T210C), NA T(1424C), NP(C182T) and M (A281G). The LAIVB with adaptive mutations is referred to herein as AM/LAIVB/DelNS1. The AM/LAIVB/DelNS1 preferably, is not able to replicate in interferon-competent cells, for example, A549 cells, and preferably replicates at low temperatures such as temperatures below 37° C., more preferably between 30 and 33° C. and most preferably, at about 33° C. A preferred LAIVB is DeINS1-B8038HK, deposited with the American Type Culture Collection, and has ATCC Deposit No. PTA-125209. The LAIVA is preferably based on an H1N1 influenza virus genome that includes a deletion of a virulence factor activity, a first set of one or more mutation(s) that confers replication at 37° C. in the absence of the virulence factor activity, and a second set of one or more mutation(s) that confers replication at a temperature below 35° C. The deletion of virulence factor activity can include a deletion of at least part of a virulence factor gene. Such a deletion can be a deletion of at least part of an NS1 gene extending beyond nucleotides 57 to 528 of an NS1 segment of the mutated virus. A preferred LAIVA is the CA4-DelNS 1, which include one or more point mutation(s) that confer replicative competence, which can lie outside of an M region of the mutated H1N1 influenza virus (for example, a G346A mutation in the H1N1 influenza virus genome). The second set of one or more mutation(s) can include one or more point mutation(s), such as a T261G or an A310G mutation in the H1N1.

The LAIVA, LAIVB, and chimeric A/B-Strain-DelNS1-Sars-CoV-2-CoV2Ag preferably replicate at low temperatures such as temperatures below 37° C. more preferably between 30 and 33° C. and most preferably, at about 33° C. In some embodiments, the disclosed chimeric viruses A/B-Strain-DelNS1-Sars-CoV-2-CoV2Ag are characterized in that they replicate poorly in MDCK cells at 37° C., when compared to its replication at 33° C. in the MDCK cells. In a particularly preferred embodiment, chimeric A/B-Strain-DelNS1-Sars-CoV-2-CoV2Ag are able to replicate at levels comparable to wild type influenza virus of the same strain, in a vaccine producing system for example, eggs or MDCK cells.

Also disclosed are methods for making chimeric viruses expressing one or more antigens of the Sars-CoV-2. The chimeric virus strains include a LAIV A or B which includes a deletion of the viral virulence element, the NS1 protein and adaptive mutations that allows growth of the mutated strain in vaccine producing systems such as eggs and MDCK cells (i.e., chimeric A/B-Strain-DelNS1-Sars-CoV-2-CoV2Ag strains). The methods include (a) generating a LAIV (A or B) (which includes a deletion of the coding region of the NS1 coding region), DeLNS1, to form A-Strain-DeLNS1 (where the LAIV is an influenza A virus) or B-Strain-DeLNS1 (where the LAIV is an influenza B virus) for example, CA04-DelNS1 (b) expressing an antigen from Sars-CoV-2 (i.e., CoV2Ag) in the A-Strain-DeLNS1 or B-Strain-DeLNS1, for example, CA04-DelNS1 or B8038-DelNS1, by transfecting A/B-Strain-DeLNS1 to express the coronavirus antigen in the place of the deleted NS1, thereby generating a chimeric virus, herein A/B-Strain-DelNS1-Sars-CoV-2-CoV2Ag (b) rescuing the A/B-Strain-DelNS1-Sars-CoV-2-CoV2Ag and (c) passaging rescued virus in one or more vaccine producing cells until viral titer is stabilized, to obtain the A-Strain-DelNS1-Sars-CoV-2-CoV2Ag strain. Exemplary coronavirus antigen domains include receptor binding domain (RBD). Following a similar protocol used to generate the chimeric Sars-CoV-2 virus, the LAIVA/B can be engineered to replace the deleted NS1 segment, with HA or NA from a different strain or with HEF (hemagglutinin-esterase fusion).

The disclosed methods preferably include reverse genetics. In some preferred embodiments, plasmids containing the deleted NS1 segment (DelNS1) and expressing the selected coronavirus antigen and the other seven genome segments derived from an influenza virus strain, are transfected into 293T/MDCK cell mixture. Rescued virus is passaged in MDCK cells until virus titer is stabilized, with virus titer maintained without meaningful change for three consecutive passages. As used herein, without meaningful change refers to changes including no change or no statistically significant change.

Pharmaceutical compositions are also provided. The pharmaceutical compositions include the disclosed immunogenic chimeric A/B-Strain-DelNS1-Sars-CoV-2-CoV2Ag, such as CA04-DelNS1-RBD or B8038-DelNS1-RBD-, produced according to the disclosed methods alone, or in combination with LAIVA and/LAIVB and/or LAIVA/B. The pharmaceutical compositions typically include an effective amount of a virus to induce an immune response in subject in need thereof when administered to the subject. The pharmaceutical compositions can include additional agents, for example adjuvants to enhance the immune response. In some embodiments, the pharmaceutical compositions do not include an adjuvant. In one embodiment, the composition include an effective mount of the chimeric CA04-DelNS 1-CoV2Ag, DelNS1-B8038 alone or in combination with DeINS1-B8038HK.

Methods of treating a subject in need thereof by administering the pharmaceutical composition to the subject are also provided. The methods can be vaccine protocols. Thus, in some embodiments, the subject is administered the composition to provide prophylactic or therapeutic protection against Sars-CoV-2, alone or in combination with the influenza virus. The disclosed chimeric LAIV A/B-Sars-CoV-2 virus exemplified for example, CA04-DelNS1-CoV2Ag or DelNS1-B8038 generated according to the methods disclosed herein, alone or in combination with the disclosed LAIVA and/or LAIVB, are administered to a mammal in need thereof by subcutaneous (s.c.), intradermal (i.d.), intramuscular (i.m.), intravenous (i.v.), oral, or intranasal administration; or by injection or by inhalation. In other aspects, the strain is administered intranasally. The compositions containing chimeric DelNS1-CoV2Ag are administrated to a mammal in need of protective immunity against a SARS-CoV-2-infection.

### BRIEF DESCRIPTION OF THE DRAWINGS

FIGS. 1A and 1B show construction of DelNS1-MERS-RBD and DelNS1-MERS-N LAIV.

FIGS. 2A-2C Protection of DPP4 transgenic mice with inoculation of lethal challenge of MERS Coronavirus (2 MLD₅₀).

FIGS. 3A-3C Protection of DPP4 transgenic mice with inoculation of lethal challenge of MERS coronavirus (10 MLD₅₀)

FIGS. 4A and 4B show Sequences of the Receptor Binding Domain (SEQ ID NO:27) of the MERS coronavirus (FIG. 4A) and Receptor Binding Domain (SEQ ID NO:28) of SARS-CoV-2 (FIG. 4B).

FIG. 5 shows cloning of Sars-CoV-2 into DelNS1 LAIV vector.

FIG. 6 is a blot showing Verification of NS segment and RBD insert in DelNS1-Sars-CoV-2-RBD vaccine strain

FIG. 7 shows the expression of Sars-CoV-2 RBD in DelNS1-Sars-CoV-2-RBD live attenuated virus infected MDCK cells.

FIG. 8 shows protection of ACE2 transgenic from diseases caused by infection of SARS-CoV-2.

FIG. 9A is an illustration of generation of 8308B-DelNS 1 influenza B virus by reverse genetics. pHW2000 plasmids containing the DelNS1 segment and the other seven genome segments derived from B/Hong Kong/8038/2011 (Victoria) virus were transfected into 293T/MDCK cell mixture. Rescued virus was passaged in MDCK cells until virus titer was stabilized. FIG. 9B. shows identified adaptive mutations in growth adapted DelNS1-B8308 virus. Four mutations in PA(T210C), NA T(1424C), NP(C182T) and M (A281G) were identified. FIG. 9C is an illustration of construction of Flu B DelNS1-SARS-RBD LAIV vaccine. RBD derived from clinical sample of SARS-CoV2 infected patient was clone into influenza B DelNS1 LAIV vector. The recombinant NS segment together with rest of the seven segments (PB1, PB2, PA, NP, HA, NA and M) of influenza B (B/HK/8038/2011) are cloned in the reverse genetic vector and transfected into 293T/MDCK cell mixture. Rescued virus are passaged in MDCK cells first then in chicken embryonated eggs.

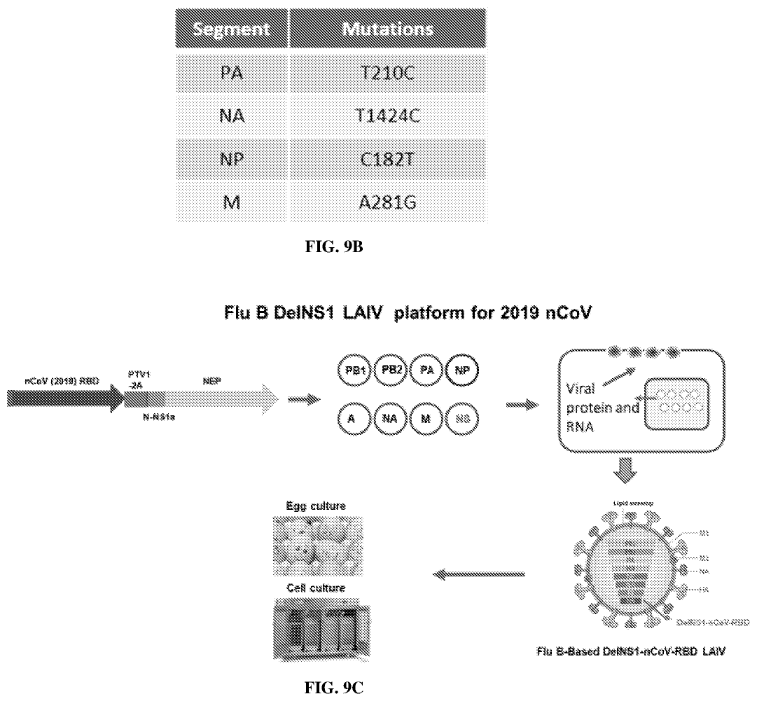

### DETAILED DESCRIPTION OF THE INVENTION

### I. Definitions

Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

As used herein, the term “adjuvant” refers to a compound or mixture that enhances an immune response.

As used herein, “attenuated” refers to refers to procedures that weaken an agent of disease (a pathogen). An attenuated virus is a weakened, less vigorous virus. A vaccine against a viral disease can be made from an attenuated, less virulent strain of the virus, a virus capable of stimulating an immune response and creating immunity but not causing illness or less severe illness. Attenuation can be achieved by chemical treatment of the pathogen, through radiation, or by genetic modification, using methods known to those skilled in the art. Attenuation may result in decreased proliferation, attachment to host cells, or decreased production or strength of toxins.

As used herein, “elderly”, refers to a subject older than 65 years of age.

As used herein, the term “effective amount” or “therapeutically effective amount” means a dosage sufficient to treat, inhibit, or alleviate one or more symptoms of a disease state being treated or to otherwise provide a desired pharmacologic effect. The precise dosage will vary according to a variety of factors such as subject-dependent variables (e.g., age, immune system health, etc.), the disease, and the age of the subject.

As used herein, the term “gene” refers to a nucleic acid (e.g., DNA or RNA) sequence that including coding sequences necessary for the production of a polypeptide, RNA (e.g., including but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein “immunogenic composition” means that the composition can induce an immune response and is therefore antigenic. By “immune response” means any reaction by the immune system. These reactions include the alteration in the activity of an organism's immune system in response to an antigen and can involve, for example, antibody production, induction of cell-mediated immunity, complement activation, or development of immunological tolerance.

As used herein, “nasal administration” refers to any form of administration whereby an active ingredient is propelled or otherwise introduced into the nasal passages of a subject so that it contacts the respiratory epithelium of the nasal cavity, from which it is absorbed into the systemic circulation. Nasal administration can also involve contacting the olfactory epithelium, which is located at the top of the nasal cavity between the central nasal septum and the lateral wall of each main nasal passage. The region of the nasal cavity immediately surrounding the olfactory epithelium is free of airflow. Thus, specialized methods must typically be employed to achieve significant absorption across the olfactory epithelium.

As used herein, “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a vaccine composition from the mouth, administration of a vaccine composition through a nasojejunal or gastrostomy tube, intraduodenal administration of a vaccine composition, and rectal administration, e.g., using suppositories that release a live bacterial vaccine strain described herein.

As used herein, “mammal” includes both humans and non-humans and include but is not limited to humans, non-human primates, canines, felines, murines, bovines, equines, and porcines.

As used herein, “topical administration” refers to the application of a pharmaceutical agent to the external surface of the skin or the mucous membranes (including the surface membranes of the nose, lungs and mouth), such that the agent crosses the external surface of the skin or mucous membrane and enters the underlying tissues. Topical administration can result in a limited distribution of the agent to the skin and surrounding tissues or, when the agent is removed from the treatment area by the bloodstream, systemic distribution of the agent. In a preferred form, the agent is delivered by transdermal delivery, e.g., using a transdermal patch. Transdermal delivery refers to the diffusion of an agent across the skin (stratum corneum and epidermis), which acts as a barrier few agents are able to penetrate. In contrast, the dermis is permeable to absorption of many solutes and drugs, and topical administration therefor occurs more readily through skin which is abraded or otherwise stripped of the epidermis to expose the dermis. Absorption through intact skin can be enhanced by combining the active agent with an oily vehicle (e.g., creams, emollients, penetration enhancers, and the like, as described, e.g., in Remington's Pharmaceutical Sciences, current edition, Gennaro et al., eds.) prior to application to the skin (a process known as inunction).

As used herein, the term “peptide” refers to a class of compounds composed of amino acids chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides.

As used herein “chimeric virus” refers to a virus stain including viral RNA from more than one type of viral strain.

As used herein, a “variant,” “mutant,” or “mutated” polynucleotide or polypeptide contains at least one polynucleotide or polypeptide sequence alteration as compared to the polynucleotide or polypeptide sequence of the corresponding wild-type or parent polynucleotide or polypeptide. Mutations may be natural, deliberate, or accidental. Mutations include substitutions, deletions, and insertions.

### II. Compositions

Immunogenic compositions including live attenuated chimeric virus are provided, based on DelNS1 live attenuated influenza A or B virus (LAIVA or LAIV B) containing a deleted NS1 segment (DelNS1), and engineered to express one or more antigens from Sar-CoV-2 (herein, CoV2Ag) in place of the deleted NS1 segment.

The compositions include the chimeric viruses alone, or as co-compositions with a LAIVA and/or LAIVB, which in some embodiments, are engineered to express various hemagglutinin (HA) and neuraminidase (NA) of influenza. Influenza viruses are classified into three types: A, B, and C. The genomes of A- and B-type influenza viruses consist of eight RNA segments, whereas influenza C viruses only have seven RNAs. Both A and B influenza viruses contain two major surface glycoproteins: the hemagglutinin (HA) and the neuraminidase (NA). Influenza C viruses have only one major surface glycoprotein, HEF (hemagglutinin-esterase fusion). Influenza A viruses are divided into subtypes based their hemagglutinin (H) and the neuraminidase (N). There are 18 different hemagglutinin subtypes (H1-H18) and 11 different neuraminidase subtypes (N1-N11). See also Russell, et al., Trends in Microbiol. 26(10):P841-853 doi:https://doi.org/10.1016/j.tim.2018.03.005 (2018), which is specifically incorporated by references herein in its entirety. See also McAuley, et al., Front Microbiol. 10: 39, doi: 10.3389/fmicb.2019.00039 (2019), which is specifically incorporated by references herein in its entirety.

The following influenza A subtypes have affected humans, H1-H3, H5-H7, H9 and H10; N1, N2, N6-N9. H1N1 and H3N2 cause seasonal epidemics today. Influenza B viruses are not divided into subtypes, but instead are further classified into two lineages: B/Yamagata and B/Victoria. Similar to influenza A viruses, influenza B viruses can then be further classified into specific clades and sub-clades. The antigenic structures of influenza B virus HAs have been studied by sequence analysis of both naturally occurring variants and antibody-selected escape variants. See, e.g., Wang, et al., Journal of Virology, 82(6):3011-3020; DOI: 10.1128/JVI.02477-07 (2008). The nucleic acid sequences encoding influenza virus hemagglutinins and neuraminidases and the protein sequences are known, for example at the following Genbank numbers; BAA01280 (A/mallard/Alberta/35/1976); CBA17655 A/WDK/JX/12416/2005); ABV25634 (A/swine/Iowa/15/1930), AAM75158 (A/Puerto

Rico/8/1934 (Mount Sinai)); ABD62843 (A/Bellamy/1942); AAK70453 (A/Hong Kong/1035/1998); ACP41105 (A/California/04/2009), etc.

LAIVA/B can be engineered to express the NA, HA or HEF from a different influenza strain. The chimeric Sars-CoV-2 virus can be included in a formulation for administration, in a carrier, and in some embodiments, in combination with an adjuvant. The adjuvant can serve as the carrier. In some embodiments, immunogenic compositions containing the disclosed chimeric virus strains do not include an adjuvant. In some preferred embodiments, the compositions do not include full length SARS-CoV-2 spike protein or whole/complete or attenuated Sars-CoV-2.

The disclosed chimeric virus strains are based on a DelNS1 live attenuated influenza A or B virus (LAIVA or LAIVB, used interchangeably with, LAIVA/B) platform which is able to express foreign antigen from the NS1 position of NS segment of the DelNS1 LAIVA/B genome.

The compositions are immunogenic in that they can be used to elicit an immune response against the one or more CoV2Ag encoded by the LAIVA/B or against one or more influenza strains based on the engineered in NA or HA. The LAIVA/B has improved safety due to deletion of the coding region of the NS1 segment (DelNS1) and adaptive mutations (AM) which improve its growth in vaccine producing systems. Preferred chimeric influenza A viruses with these combinations of mutations which are based on a passage adapted a/ca/04/2009 (CA04) virus are referred to herein as CA04-DelNS1-CoV2Ag, when engineered to express an antigen from the SARS-CoV-2. Preferred chimeric influenza B viruses with these combinations of mutations which are based on the 8308B-DelNS1 virus strain, are referred to herein as 8308B-DelNS1-CoV2Ag when engineered to express an antigen from the SARS-CoV-2.

A. Live Attenuated Chimeric Virus

The disclosed chimeric viruses can contain various LAIV backbones containing a deleted NS1 segment (DelNS1), engineered to express one or more antigens from the novel coronavirus (herein, CoV2Ag). The resulting chimeric virus resulting from DelNS1 live attenuated influenza A or B virus (LAIVA/B), and expressing a CoV2Ag, is referred to generally herein, as DelNS1-A/B Sars-CoV-2-CoV2Ag.

(i) LAIV Backbones from Influenza A

In some embodiments, the backbone virus used to make the disclosed chimeric SARS-CoV-2 are preferably live attenuated influenza A virus strains. Exemplary strains include CA04, and A/WSN/33 and A/PR/8/34. HK4801-DelNS1-SARS-CoV-2-RBD and H1N1(2019)-DelNS1-SARS-CoV-2-RBD exemplified herein can be constructed in the internal gene backbone of CA04-DelNS1 with HA and NA derived from strain of A/HK/4801/2014 (H3N2) or A/HK/2019 (H1N1).

(a) CA04-DelNS1

A preferred LAIV backbone is a mutated influenza virus disclosed in Publication No. 20190125858, incorporated herein by reference. Briefly, cold adapted influenza virus CA04-DelNS1 is based on the 2009 H1N1 influenza stains, and accordingly, includes the that includes a deletion of a virulence factor activity, a first set of one or more mutation(s) that confers replication at 37° C. in the absence of the virulence factor activity, and a second or third set of one or more mutation(s) that confers replication at a temperature below 35° C. The deletion of virulence factor activity can include a deletion of at least part of a virulence factor gene. Such a deletion can be a deletion of at least part of an NS1 gene extending beyond nucleotides 57 to 528 of an NS1 segment of the mutated virus.

The first set of one or more point mutation(s) confer replicative competence, and can lie outside of an M region of the mutated H1N1 influenza virus (for example, a G346A (D101N in protein sequence) mutation in the H1N1 influenza virus genome).

The second set of one or more mutation(s) can include one or more point mutation(s), such as a T261G (L79V in protein sequence) or an A310G (E95G in the protein sequence) mutation in the H1N1 influenza virus genome, positions that have been found to support cold adapted DelNS1 virus replication. The disclosed mutated influenza virus can also include a third set of one or more mutation(s) that confers replication at a temperature below 35° C. These can include one or more point mutation(s) that are distinct from the second set of mutation(s), such as a T261G or an A310G mutation in the H1N1 influenza virus genome. The mutated influenza virus can show reduced replicative ability, relative to a temperature of 35° C. or lower, at a temperature of 37° C. or higher.

(b) A/WSN/33-DelNS1 and A/PR/8/34-DELNS1

The LAIV backbone can be also derived from the A/WSN/33 and A/PR/8/34 strains described in Zheng, et al., J. Virol., 89:10273-10285 (2015). These viral strains include a deletion of the NS1 gene, and an adaptive substitution, A 14U (obtained after a few passages of DelNS1 virus), in the 3′ noncoding region (NCR) of the M segment of viral RNA (vRNA) significantly enhances the replication of DelNS1 viruses. The M-A14U substitution supports PR8 DelNS1 virus replication in Vero and MDCK cells, while PR8 DelNS1 virus without this substitution cannot be propagated.

(ii) LAIV Backbones from Influenza B

The backbone virus used to make the disclosed chimeric SARS-CoV-2 in some embodiments employ live attenuated influenza B virus strains.

The influenza B virus genomes each include eight negative-sense, single-stranded viral RNA (vRNA) segments. The smallest vRNA segment of both viruses encodes the non-structural NS1 protein. Influenza B virus expresses from an unspliced transcript of the viral NS segment a 281-amino-acid nonstructural protein termed NS1-B. The LAIVB used to make the disclosed chimeric viruses preferably includes a complete deletion of the coding region for NS1 from the LAIVB genome. The AM/LAIVB/DelNS1 can be of the Yamagata or Victoria lineage. During 1988-1989 two highly distinct antigenic variants of influenza type B were recognized in hemagglutination-inhibition tests with post infection ferret serum. These viruses were antigenically related to either B/Victoria/2/87, the most recent reference strain, or B/Yamagata/16/88, a variant that was isolated in Japan in May 1988. All influenza B viruses isolated in the United States during an epidemic in the winter of 1988-1989 were antigenically related to B/Victoria/2/87. Different strains of influenza B virus are disclosed in the Influenza Research Database. Zhang, et al., Nucleic Acids Research, Volume 45(D1): D466—D474, 2017.

The eight segments of influenza B viruses (and are numbered in order of decreasing length (TABLE 1). Segments 1, 3, 4, and 5 encode just one protein per segment: the PB2, PA, HA and NP proteins.

TABLE 1
Segments of Influenza B virus and their encoded proteins

| | Encoded | |
|---|---|---|
| Segment | Protein | Protein Function |
| 1 (SEQ ID NO: 1) | PB2 | Polymerase subunit; mRNA cap recognition |
| 2 (SEQ ID NO: 2) | PB1 | Polymerase subunit; RNA elongation, endonuclease |
| | | activity |
| 3 (SEQ ID NO: 3) | PA | Polymerase subunit; protease activity |
| 4 (SEQ ID NO: 4) | HA | Surface glycoprotein; major antigen, receptor binding and |
| | | fusion activities |
| 5 (SEQ ID NO: 5) | NP | RNA binding protein; nuclear import regulation |
| 6 (SEQ ID NO: 6) | NA | Surface glycoprotein; sialidase activity, virus release |
| | NB | an integral membrane protein corresponding to the |
| | | influenza A virus M2 protein |
| 7 (SEQ ID NO: 7) | M1 | Matrix protein; vRNP interaction, RNA nuclear export |
| | | regulation, viral budding |
| 8 (SEQ ID NO: 8) | NS1 | Interferon antagonist protein; regulation of host gene |
| | | expression |
| | NEP/NS2 | Nuclear export of RNA |

All influenza viruses encode the polymerase subunit PB1 on segment 2.

PB2
(SEQ ID NO: 1)
AGTACTGGTCGACCTCCGAAGTTGGGGGGGAGCAGAAGCGGAGCGTTTTCAAGATG
ACATTGGCCAAAATTGAATTGTTAAAACAACTGCTAAGGGACAATGAAGCCAAAAC
AGTTTTGAAGCAAACAACAGTAGACCAATATAACATAATAAGAAAATTCAATACAT
CAAGGATTGAAAAGAATCCTTCACTAAGGATGAAGTGGGCCATGTGTTCTAATTTTC
CCTTGGCTCTAACCAAGGGCGATATGGCAAACAGAATCCCCTTGGAATACAAAGGG
ATACAACTCAAAACAAATGCTGAAGACATAGGAACCAAAGGCCAAATGTGCTCAAT
AGCAGCAGTTACTTGGTGGAATACATATGGACCAATAGGAGATACTGAAGGTTTCG
AAAGGGTCTACGAAAGCTTTTTTCTCAGAAAAATGAGACTTGACAACGCCACTTGGG
GCCGAATAACTTTTGGCCCAGTTGAAAGAGTGAGAAAAAGGGTACTGCTAAACCCT
CTCACCAAGGAAATGCCTCCGGATGAGGCGAGCAATGTGATAATGGAAATATTGTT
CCCTAAAGAAGCAGGAATACCAAGAGAATCCACTTGGATACATAGGGAACTGATAA
AAGAAAAAAGAGAAAAATTGAAAGGAACAATGATAACTCCAATCGTACTGGCATAC
ATGCTTGAAAGAGAACTGGTTGCTCGAAGAAGATTCTTGCCAGTGGCAGGAGCAAC
ATCAGCTGAGTTCATAGAAATGCTACACTGCTTACAAGGTGAAAATTGGAGACAAA
TATATCACCCAGGAGGGAATAAATTAACTGAGTCCAGGTCTCAATCAATGATAGTA
GCTTGTAGAAAAATAATCAGAAGATCAATAGTCGCTTCAAACCCACTGGAGCTAGC
TGTAGAAATTGCAAACAAGACTGTGATAGATACTGAACCTTTAAAGTCATGTCTGGC
AGCCATAGACGGAGGTGATGTAGCTTGTGACATAATAAGAGCTGCATTAGGACTAA
AGATCAGACAAAGACAAAGATTTGGACGGCTTGAGCTAAAAAGAATATCAGGAAG
AGGATTCAAAAATGATGAAGAAATATTAATAGGGAACGGAACAATACAGAAGATTG
GAATATGGGACGGGGAAGAGGAGTTCCATGTAAGATGTGGTGAATGCAGGGGAATA
CTAAAAAAGAGTAAAATGAAACTGGAAAAACTACTGATAAATTCAGCCAAAAAGG
AGGATATGAGAGATTTAATAATCTTATGCATGGTATTTTCTCAAGACACTAGGATGT
TCCAAGGGGTGAGAGGAGAAATAAATTTTCTTAATCGAGCAGGCCAACTTTTATCTC
CAATGTACCAACTCCAACGATATTTTTTGAATAGAAGCAACGACCTTTTTGATCAAT
GGGGGTATGAGGAATCACCCAAAGCAAGTGAACTACATGGGATAAATGAATCAATG
AATGCATCTGACTATACATTGAAAGGGATTGTAGTGACAAGAAATGTAATTGACGA
CTTTAGCTCTACTGAAACAGAAAAAGTATCCATAACAAAAAATCTTAGTTTAATAAA
AAGGACTGGGGAAGTCATAATGGGAGCTAATGACGTGAGTGAATTAGAATCACAAG
CACAGCTGATGATAACATATGATACACCTAAAATGTGGGAAATGGGAACAACCAAA
GAACTGGTGCAAAACACTTATCAATGGGTGCTAAAAAACTTGGTGACACTGAAGGC
TCAGTTTCTTCTAGGAAAAGAGGACATGTTCCAATGGGATGCATTTGAAGCATTTGA
GAGCATAATTCCTCAGAAGATGGCTGGTCAATACAGTGGATTTGCAAGAGCAGTGC
TCAAACAAATGAGAGACCAGGAGGTTATGAAAACTGACCAGTTCATAAAGTTGTTG
CCTTTTTGTTTCTCACCACCAAAATTAAGGAGCAATGGGGAGCCTTATCAATTCTTA
AAACTTGTGTTGAAAGGAGGAGGGGAAAATTTCATCGAAGTAAGGAAAGGGTCCCC
TCTATTTTCCTATAATCCACAAACAGAAGTCCTAACTATATGCGGCAGAATGATGTC
ATTAAAAGGGAAAATTGAAGATGAAGAAAGGAATAGATCAATGGGGAATGCAGTA
TTAGCAGGCTTTCTCGTTAGTGGCAAATATGACCCAGATCTTGGAGATTTCAAAACT
ATTGAAGAACTTGAAAAGCTGAAACCAGGGGAAAAGGCAAACATCTTACTTTATCA
AGGAAAACCAGTTAAAGTAGTTAAAAGGAAAAGGTATAGTGCTTTGTCCAATGACA
TTTCACAAGGAATTAAGAGACAAAGAATGACAGTTGAGTCCATGGGGTGGGCCTTG
AGCTAATATAAATTTATCCATTAATTCAATGAACGCAATTGAGTGAAAAATGCTCGT
GTTTCTACT
PB1
(SEQ ID NO: 2)
AGCAGAAGCGGAGCCTTTAAGATGAATATAAATCCTTATTTTCTCTTCATAGATGTG
CCCGTACAGGCAGCAATTTCAACAACATTCCCATACACTGGTGTTCCCCCTTATTCTC
ATGGAACAGGAACAGGCTACACAATAGACACCGTGATCAGAACGCATGAGTACTCA
AACAAGGGGAAACAGTACATTTCTGATGTTACAGGATGCACAATGGTAGATCCAAC
AAATGGACCATTACCCGAAGATAATGAGCCGAGTGCCTATGCGCAATTAGATTGCG
TTTTAGAGGCTTTGGATAGAATGGATGAAGAACACCCAGGTCTTTTTCAAGCAGCCT
CACAGAATGCTATGGAGGCCCTAATGGTCACAACTGTAGACAAATTAACCCAGGGG
AGACAGACTTTTGATTGGACAGTATGCAGAAACCAACCTGCTGCAACGGCACTGAA
CACAACAATAACCTCTTTTAGGTTGAATGATTTAAATGGAGCCGACAAAGGTGGATT
AATACCTTTTTGTCAGGATATCATTGATTCATTAGACCGACCTGAAATGACTTTCTTC
TCAGTAAAGAATATAAAGAAAAAATTGCCTGCCAAAAACAGAAAGGGTTTCCTCAT
AAAGAGGATACCAATGAAGGTAAAAGACAAAATAACCAAAGTGGAATACATCAAA
AGAGCATTATCATTAAACACAATGACAAAAGACGCTGAAAGAGGCAAATTGAAAAG
AAGAGCGATTGCCACTGCTGGAATACAAATCAGAGGGTTTGTATTAGTAGTTGAAA
ACTTGGCTAAAAATATATGTGAAAATCTAGAACAAAGTGGTTTACCAGTAGGTGGA
AACGAGAAGAAAGCCAAACTGTCAAACGCAGTGGCCAAAATGCTCAGTAACTGCCC
ACCAGGAGGGATTAGCATGACAGTAACAGGAGACAATACAAAATGGAATGAATGTT
TAAACCCAAGAATCTTTTTGGCTATGACTGAAAGAATAACCAGAGACAGCCCAATTT
GGTTCAGGGATTTTTGTAGTATAGCACCGGTCCTGTTCTCCAATAAGATAGCAAGAT
TGGGGAAAGGGTTTATGATAACAAGCAAAACAAAAAGACTGAAGGCTCAAATACCT
TGTCCTGATCTGTTTAGTATACCATTAGAAAGATATAATGAAGAAACAAGGGCAAA
ATTGAAAAAGCTAAAACCATTCTTCAATGAAGAAGGAACTGCATCTTTGTCGCCTGG
GATGATGATGGGAATGTTTAATATGCTATCTACCGTGTTGGGAGTAGCTGCACTAGG
TATCAAGAACATTGGAAACAAAGAATACTTATGGGATGGACTGCAATCTTCTGATG
ATTTTGCTCTATTTGTTAATGCAAAGGATGAAGAAACATGTATGGAAGGAATAAACG
ACTTTTACCGAACATGTAAATTATTGGGAATAAACATGAGCAAAAAGAAAAGTTAC
TGTAATGAGACTGGAATGTTTGAATTTACAAGCATGTTCTACAGAGATGGATTTGTA
TCTAATTTTGCAATGGAACTCCCTTCGTTTGGGGTTGCTGGAGTAAATGAATCAGCA
GATATGGCAATAGGAATGACAATAATAAAGAACAACATGATCAACAATGGGATGGG
TCCAGCAACAGCACAAACAGCCATACAGTTATTCATAGCTGATTATAGATACACCTA
CAAATGCCACAGGGGAGATTCCAAAGTAGAAGGAAAGAGAATGAAAATCATAAAG
GAGTTATGGGAAAACACTAAAGGAAGAGATGGTCTATTAGTAGCAGATGGTGGGCC
CAACATTTACAATTTGAGAAACTTGCATATCCCAGAAATAGTATTAAAGTATAATCT
AATGGACCCTGAATACAAAGGGCGGTTACTTCATCCTCAAAATCCCTTTGTGGGACA
TTTGTCTATTGAAGGCATCAAAGAGGCAGATATAACCCCAGCACATGGTCCAGTAA
AGAAAATGGACTACGATGCGGTGTCTGGAACTCATAGTTGGAGAACCAAAAGAAAC
AGATCTATACTAAACACTGATCAGAGGAACATGATTCTTGAGGAACAATGCTACGCT
AAATGTTGCAACCTATTTGAGGCCTGTTTTAACAGTGCATCATACAGGAAGCCAGTG
GGTCAACATAGCATGCTTGAGGCTATGGCCCACAGATTAAGAATGGATGCACGATT
AGATTATGAATCAGGGAGAATGTCAAAGGATGATTTTGAGAAAGCAATGGCTCACC
TTGGTGAGATTGGGTACATATAAGCTTCGAAGATGTTTATGGGGTTATTGGTCACCA
TTGAATACATGCGATACACAAATGATTAAAATGAAAAAAGGCTCGTGTTTCTACT
PA
(SEQ ID NO: 3)
AGCAGAAGCGGTGCGTTTGATTTGTCATAATGGATACTTTTATTACAAGAAACTTCC
AGACTACAATAATACAAAAGGCCAAAAACACAATGGCAGAATTTAGTGAAGATCCT
GAATTGCAACCAGCAATGCTATTCAATATCTGCGTCCATCTAGAGGTTTGCTATGTA
ATAAGTGACATGAATTTTCTTGACGAAGAAGGAAAAGCACATATAGCATTAGAAGG
ACAAGGGAAAGAACAAAACTTGAGACCACAATATGAAGTAATTGAGGGAATGCCA
AGAACCATAGCATGGATGGTCCAGAGATCCTTAGCTCAAGAGCATGGAATAGAGAC
TCCCAAGTATCTGGCTGATTTGTTTGATTATAAAACCAAAAGATTTATAGAAGTTGG
AATAACAAAAGGATTGGCTGATGATTACTTTTGGAAAAAGAAAGAAAAGTTGGGAA
ATAGCATGGAACTGATGATATTCAGCTACAATCAAGACTACTCGTTAAGTAATGAAT
CCTCATTGGATGAGGAAGGGAAAGGGAGAGTGCTAAGCAGACTCACAGAACTTCAG
GCTGAGTTAAGTCTGAAAAACTTATGGCAAGTTCTCATAGGAGAGGAAGATGTTGA
AAAGGGAATTGATTTTAAACTTGGACAAACAATATCTAGACTAAGGGATATATCTGT
TCCGGCTGGTTTTTCCAATTTTGAAGGAATGAGGAGCTACATAGACAATATAGACCC
AAAAGGAGCAATAGAGAGAAATCTAGCAAGGATGTCTCCCTTAGTATCAGTCACAC
CTAAAAAGTTAACATGGGAGGACCTAAGACCGATAGGGCCTCACATTTATGACCAT
GAGCTACCAGAAGTTCCATATAATGCCTTTCTTCTAATGTCTGATGAACTGGGACTG
GCCAATATGACTGAGGGAAAATCCAAAAAACCGAAGACATTAGCCAAAGAATGTCT
AGAAAAGTACTCAACACTACGGGATCAAACTGACCCAATATTAATAATGAAAAGCG
AAAAAGCTAACGAAAATTTCCTATGGAAGCTTTGGAGAGACTGTGTAAACACAATA
AGTAATGAGGAAACAAGTAACGAGTTACAGAAAACCAATTATGCCAAATGGGCTAC
AGGGGACGGATTAACATACCAGAAAATAATGAAAGAAGTAGCAATAGATGACGAA
ACAATGTGCCAAGAAGAGCCTAAAATCCCTAACAAATGTAGAGTGGCTGCTTGGGT
TCAAACAGAGATGAATCTATTGAGCACTCTGACAAGTAAAAGAGCTCTGGACCTAC
CAGAAATAGGGCCAGACATAGCACCCGTGGAGCATGTAGGAAGTGAAAGAAGGAA
ATACTTTGTTAATGAAATCAACTACTGTAAGGCCTCTACAGTTATGATGAAGTATGT
GCTTTTTCACACTTCATTGTTGAATGAAAGCAATGCCAGCATGGGAAAATACAAAGT
AATACCAATAACCAATAGAGTAGTAAATGAAAAAGGAGAAAGTTTCGACATGCTTT
ACGGTCTAGCGGTTAAAGGACAATCTCATCTGAGGGGAGATACTGATGTTGTAACA
GTTGTAACTTTCGAATTTAGTGGTACAGATCCAAGAGTGGACTCAGGAAAGTGGCCA
AAATATACTGTGTTTAGGATTGGCTCCCTATTTGTGAGTGGGAGGGAAAAATCTGTG
TACTTGTATTGCCGAGTGAATGGCACAAATAAGATCCAAATGAAATGGGGAATGGA
AGCTAGAAGATGTTTGCTTCAATCAATGCAACAAATGGAGGCAATTGTTGAACAGG
AATCATCAATACAAGGATATGACATGACCAAAGCCTGTTTCAAGGGAGACAGAGTA
AATAGCCCCAAAACTTTCAGTATTGGAACTCAAGAAGGAAAACTAGTAAAAGGATC
CTTTGGAAAAACACTAAGAGTAATATTTACTAAATGCTTGATGCACTATGTATTTGG
AAATGCCCAATTGGAGGGGTTTAGTGCCGAGTCTAGGAGACTTCTACTGTTGATTCA
AGCATTAAAGGACAGAAAGGGCCCCTGGGTGTTCGACTTAGAGGGAATGTATTCTG
GAATAGAAGAATGTATTAGCAACAACCCTTGGGTAATACAGAGTGTATACTGGTTC
AATGAATGGTTGGGCTTTGAAAAGGAGGGGAGTAAAGTGTTGGAATCAGTGGATGA
AATAATGGATGAATAAAAGGAAATGGTACTCAATTTGGTACTATTTTGTTCATTATG
TATCTAAACATCCAATAAAAAGAACCAAGAATCAAAAATGCACGTGTTTCTACT
HA
(SEQ ID NO: 4)
AGCAGAAGCAGAGCATTTTCTAATATCCACAAAATGAAGGCAATAATTGTACTACTC
ATGGTAGTAACATCCAATGCAGATCGAATCTGCACTGGGATAACATCGTCAAACTCA
CCACATGTCGTCAAAACTGCTACTCAAGGGGAGGTCAATGTGACTGGTGTAATACCA
CTGACAACAACACCCACCAAATCTCATTTTGCAAATCTCAAAGGAACAGAAACCAG
AGGGAAACTATGCCCAAAATGCCCCAACTGCACAGATCTGGACGTAGCCTTGGGCA
GACCAAAATGCACGGGAAAAATACCCTCGGCAAGAGTTTCAATACTCCATGAAGTC
AGACCTGTTACATCTGGGTGCTTTCCTATAATGCACGACAGAACAAAAATTAGACAG
CTGCCTAACCTTCTCCGAGGATACGAACATATCAGATTATCAACTCATAACGTTATC
AATGCAGAAAGTGCACCAGGAGGACCCTACAAAATTGGAACCTCAGGGTCTTGCCC
TAACGTTACCAATGGAAACGGATTTTTCGCAACAATGGCTTGGGCCGTCCCAAAAAA
CGACAAAAACAAAACAGCAACAGATCCATTAACAATAGAAGTACCATACATTTGTA
CAGAAGGAGAAGACCAAATTACCGTTTGGGGGTTCCACTCTGATAACGAGATCCAA
ATGGCAAAGCTCTATGGGGACTCAAAGCCCCAGAAGTTCACCTCATCTGCCAACGG
AGTGACCACACATTACGTTTCACAGATTGGTGGCTTCCCAAATCAAACAGAAGACG
GAGGACTACCACAAAGTGGTAGAATTGTTGTTGATTACATGGTACAAAAATCTGGG
AAAACAGGAACAATTACCTATCAAAGAGGTATTTTATTGCCTCAAAAGGTGTGGTGC
GCAAGTGGCAGGAGCAAGGTAATAAAAGGATCCTTGCCTTTAATTGGAGAAGCAGA
TTGCCTCCACGAAAAATACGGTGGATTAAACAAAAGCAAGCCTTACTACACAGGGG
AACATGCAAAGGCCATAGGAAATTGCCCAATATGGGTGAAGACACCCTTGAAGCTG
GCCAATGGAACCAAATATAGACCTCCTGCAAAACTATTAAAGGAAAGGGGTTTCTT
CGGAGCTATTGCTGGTTTCTTAGAAGGAGGATGGGAAGGAATGATTGCAGGTTGGC
ACGGATACACGTCCCATGGGGCACATGGAGTAGCGGTGGCAGCAGACCTTAAGAGC
ACTCAAGAGGCCATAAACAAGATAACAAAAAATCTAAACTCTTTGAGTGAGCTGGA
AGTAAAGAATCTTCAAAGACTAAGCGGTGCCATGGATGAACTCCACAACGAAATAC
TAGAACTAGACGAGAAAGTAGATGATCTCAGAGCTGATACAATAAGCTCACAAATA
GAACTCGCAGTCCTGCTTTCCAATGAAGGAATAATAAACAGTGAAGATGAACATCT
CTTGGCGCTTGAAAGAAAGCTGAAGAAAATGCTGGGGCCCTCTGCTGTAGAGATAG
GGAATGGATGCTTTGAAACCAAACACAAGTGCAACCAGACCTGTCTCGACAGAATA
GCTGCTGGTACCTTTGACGCAGGAGAATTTTCTCTCCCCACCTTTGATTCACTGAATA
TTACTGCTGCATCTTTAAATGACGATGGATTGGATAATCATACCATACTGCTTTACTA
CTCAACTGCTGCCTCCAGTTTGGCTGTAACACTGATGATAGCTATCTTTGTTGTTTAT
ATGGTCTCCAGAGACAATGTTTCTTGCTCCATCTGTCTATAAGGGAAGTTAAGCCCT
GTGTTTTCCTTTGTTGTAGTGCTTGTTTGCTTGTTGCCATTACAAAGAAACGTTATTG
AAAAATGCTCTTGTTACT
NP
(SEQ ID NO: 5)
AGCAGAAGCACAGCATTTTCTTGTGAACTTCAAGCACCAGTAAAAGAACTGAAAAT
CAAAATGTCCAACATGGATATTGACGGTATAAACACTGGGACAGTTGACAAAACAC
CGGAAGAAATAACTTCTGGAACCAGTGGGACAACCAGACCAATCATTAGACCAGCA
ACCCTTGCCCCACTAAGCAACAAACGAACCCGTAACCCATCCCCGGAAAGAGCAAC
TACAAGCAGTGAAGATGATGTCGGAAGGAAAACCCAAAAGAAACAGACCCCGACA
GAGATAAAGAAGAGCGTCTACAACATGGTGGTGAAACTGGGCGAATTCTATAACCA
GATGATGGTCAAAGCTGGACTCAATGATGACATGGAGAGAAACCTAATCCAAAATG
CGCATGCCGTGGAAAGAATTCTATTGGCTGCCACTGATGACAAGAAAACCGATTTCC
AGAAGAAAAAGAATGCCAGAGATGTCAAAGAAGGGAAAGAAGAAATAGATCACAA
CAAAACAGGAGGCACCTTTTATAAGATGGTAAGAGATGATAAAACCATCTACTTTA
GCCCTATAAGAATTACCTTTTTAAAAGAAGAGGTGAAAACAATGTACAAAACCACC
ATGGGGAGTGATGGCTTCAGTGGACTAAATCACATAATGATTGGGCATTCACAGAT
GAATGATGTCTGTTTCCAAAGATCAAAGGCACTAAAAAGAGTTGGACTTGATCCTTC
ATTAATCAGTACCTTTGCGGGAAGCACAGTCCCCAGAAGATCAGGTGCGACTGGTGT
TGCAATCAAAGGAGGTGGAACTTTAGTGGCTGAAGCCATTCGATTTATAGGAAGAG
CAATGGCAGACAGAGGGCTATTGAGAGATATCAAAGCCAAGACTGCCTATGAAAAG
ATTCTTCTGAATCTAAAAAACAAATGCTCTGCGCCTCAACAAAAGGCTCTAGTTGAT
CAAGTGATCGGAAGCAGAAATCCGGGGATTGCAGACATTGAAGATCTAACCCTGCT
TGCTCGTAGTATGGTCGTTGTTAGGCCCTCTGTGGCAAGCAAAGTGGTGCTTCCCAT
AAGCATTTACGCCAAAATACCTCAACTAGGGTTCAATGTTGAAGAGTACTCTATGGT
TGGGTACGAAGCCATGGCTCTTTACAATATGGCAACACCTGTGTCCATATTAAGAAT
GGGAGATGATGCAAAAGATAAATCGCAATTATTCTTCATGTCTTGCTTCGGAGCTGC
CTATGAAGACCTGAGAGTTTTGTCTGCATTGACAGGCACAGAATTCAAACCTAGATC
AGCATTAAAATGCAAGGGTTTCCATGTTCCAGCAAAGGAACAAGTAGAAGGAATGG
GAGCAGCTCTGATGTCCATCAAGCTCCAGTTTTGGGCTCCAATGACCAGATCTGGGG
GGAACGAAGTAGGTGGAGACGGAGGGTCTGGCCAAATAAGCTGCAGCCCAGTGTTT
GCAGTGGAAAGACCTATTGCTCTAAGCAAGCAAGCTGTAAGGAGAATGCTATCAAT
GAATATTGAGGGACGTGATGCAGATGTCAAAGGAAATCTACTCAAGATGATGAATG
ACTCAATGGCTAAGAAAACCAGTGGAAATGCTTTCATTGGGAAGAAAATGTTTCAA
ATATCAGACAAAAACAAAACCAATCCCATTGAAATTCCAATTAAGCAGACCATCCC
CAATTTCTTCTTTGGGAGGGACACAGCAGAGGATTATGATGACCTCGATTATTAAAG
CAACAAAATAGACACTATGACTGTGATTGTTTCAATACGTTTGGAATGTGGGTGTTT
ATTCTTATTAAAATAAATATAAAAAATGCTGTTGTTTCTACT
NA
(SEQ ID NO: 6)
AGCAGAAGCAGAGCATCTTCTCAAAATTGAAGCAAATAGGCCGAAAATGAACAATG
CTACCCTCAACTATACAAACGTTAACCCTATTTCTCACATCAGGGGGAGTATTATTA
TCACTATATGTGTCAGCTTCACTGTCATACTTACTATATTCGGATATATTGCTAAAAT
TCCCATCAACAGAAATTACTGCACCAACAATGCCATTAGATTGTGCAAACGCATCAA
ATGTTCAGGCTGTGAACCGTTCTGCAACAAAAGGGGTGACACTTCTTCTCCCAGAAC
CGGAGTGGACATACCCGCGTTTATCTTGCCCGGGCTCAACCTTTCAGAAAGCACTCC
TAATTAGCCCTCATAGATTCGGAGAGACCAAAGGAAACTCAGCTCCCTTGATAATAA
GGGAACCTTTTATTGCTTGTGGACCAAAGGAATGCAAACACTTTGCTCTAACCCACT
ATGCAGCCCAACCAGGGGGATACTACAATGGAACAAGAGGAGACAGAAACAAGCT
GAGGCATCTAATTTCAGTCAAATTGGGCAAAATCCCAACAGTAGAAAACTCCATTTT
CCACATGGCAGCATGGAGCGGGTCCGCATGTCATGATGGTAAGGAATGGACATATA
TCGGAGTTGATGGCCCTGACAATAATGCATTGCTCAAAATAAAATATGGGGAAGCA
TATACTGACACATACCATTCCTATGCAAACAACATCCTAAGAACACAAGAAAGTGC
CTGCAATTGCATCGGAGGAAATTGTTATCTTATGATAACTGATGGCTCAGCTTCAGG
TGTTAGTGAATGCAGATTTCTTAAAATTCGAGAGGGCCGAATAATAAAAGAAATATT
TCCAACAGGAAGAATACAACATACTGAAGAATGCACATGCGGATTTGCTAGCAATA
AAACCATAGAATGTGCCTGTAGAGATAACAGTTACACAGCAAAAAGACCCTTTGTC
AAATTAAACGTGGAGACTGATACAGCAGAAATAAGATTGATGTGCACAAAGACTTA
TTTGGACACCCCCAGACCAGAGGATGGAAGCATAACTGGGCCTTGTGAATCTAATG
GAGGCAAAGGGAGTGGAGGCATCAAGGGAGGATTTGTCCATCAAAGAATGGCATCC
AAGATTGGAAGGTGGTACTCTCGAACGATGTCTAAAACTGAAAGGAAGGGGATGGG
GCTGTATGTCAAGTATAATGGAGACCCATGGGCTGACAGTGATGCCCTTGTTTTTAG
TGGAGTAATGGTTTCAATGGAAGAACCTGGTTGGTACTCCTTTGGCTTCGAAATAAA
AGACAAGAAATGTGATGTCCCCTGTATTGGGATAGAGATGGTACATGATGGTGGAA
AAGAGACTTGGCACTCAGCAGCTACAGCCATTTACTGTTTAATGGGCTCAGGACAGC
TGCTGTGGGACACTGCCACAGGTGTTAATATGACTCTGTAATGGAGGAATGGTTGAG
TCTGCTCTAAACCCTTTGTTCCTATTTTGTTTGAACAATTGTCCTTACTAAACTTAATT
GTTTCTGAAAAATGCTCTTGTTACTACT
M
(SEQ ID NO: 7)
AGCAGAAGCAGGCACTTTCTTAAAATGTCGCTGTTTGGAGACACAATTGCCTACTTG
CTTTCATTGACAGAAGATGGAGAAGGCAAACCAGAACTAGCAGAAAAATTACACTG
TTGGTTTGGTGGGAAAGAATTTGACCTAGACTCTGCCTTAGAATGGATAAAAAACAA
AAGATGCTTAACTGATATACAAAAAGCACTAATTGGTGCCTCTATATGCTTTTTAAA
ACCCAAAGACCAGGAAAGAAAAAGAAGATTCATCACAGAGCCCTTATCAGGAGTGG
GAACAACAGCAACAAAAAAGAAAGGCCTGATTCTGGCTGAGAGAAAAATGAGAAG
ATGTGTGAGCTTTCATGAAGCATTTGAAATAGCAGAAGGCCATGAAAGCTCAGCGC
TACTATACTGTCTCATGGTCATGTACCTGAATCCTGGAAATTATTCAATGCAAGTAA
AACTAGGAACGCTCTGTGCTTTGTGCGAGAAACAAGCATCACATTCACACAGAGCTC
ATAGCAGAGCAGCGAGATCTTCAGTGCCTGGAGTGAGACGAGAAATGCAGATGGTC
TCAGCTATGAACACAGCAAAAACAATGAATGGAATGGGAAAAGGAGAAGACGTCC
AAAAGCTGGCAGAAGAGCTGCAAAGCAACATTGGAGTGCTGAGATCTCTTGGGGCA
AGTCAAAAGAATGGGGAAGGAATTGCAAAGGATGTAATGGAAGTGCTAAAGCAGA
GCTCTATGGGAAATTCAGCTCTTGTGAAGAAATATCTATAATGCTCGAACCATTTCA
GATTCTTTCAATTTGTTCTTTTATCTTATCAGCTCTCCATTTCATGGCTTGGACAATAG
GGCATTTGAATCAAATAAAAAGAGGAATAAACATGAAAATACGAATAAAAAGTCCA
AACAAAGAGACAATAAACAGAGAGGTATCAATTTTGAGACACAGTTACCAAAAAGA
AATCCAGGCCAAAGAAACAATGAAGGAAGTACTCTCTGACAACATGGAGGTATTGA
GTGACCACATAATAATTGAGGGGCTTTCTGCCGAAGAAATAATAAAAATGGGTGAA
ACAGTTTTGGAGATAGAAGAATTGCATTAAATTCAATTTTTGCTGTATTTCTTACTAT
GCATTTAAGCAAATTGTAATCAATGTCAGCAAATAAACTGGAAAAAGTGCGTTGTTT
CTACT
NS
(SEQ ID NO: 8)
AGCAGAAGCAGAGGATTTGTTTAGTCACTGGCAAACAGGAAAAATGGCGAACAACA
TGACCACAACACAAATTGAGGTGGGTCCGGGAGCAACCAATGCCACCATAAACTTT
GAAGCAGGAATTCTGGAGTGCTATGAAAGGCTTTCATGGCAAAGAGCCCTTGACTA
CCCTGGTCAAGACCGCCTAAACAGACTAAAGAGAAAATTAGAGTCAAGAATAAAGA
CTCACAACAAAAGTGAGCCTGAAAGTAAAAGGATGTCCCTTGAAGAGAGAAAAGCA
ATTGGAGTAAAAATGATGAAAGTACTCCTATTTATGGATCCGTCTGCTGGAATTGAA
GGGTTTGAGCCATACTGTATGAAAAGTTCCTCAAATAGCAACTGTACGAAATACAAT
TGGACCGATTACCCTTCAACACCAGGGAGATGCCTTGATGACATAGAAGAAGAACC
AGAGGATGTTGATGGCCCCACTGAAATAGTATTAAGGGACATGAACAACAAAGATG
CGAGGCAAAAGATAAAGGAGGAAGTAAACACTCAGAAAGAAGGGAAGTTCCGTTT
GACAATAAAAAGGGATATGCGTAATGTATTGTCCTTGAGAGTGTTGGTAAACGGAA
CATTCCTCAAACATCCCAATGGATACAAGTCCTTATCAACTCTGCATAGATTGAATG
TATATGACCAGAGTGGAAGGCTTGTTGCTAAACTTGTTGCTACTGATGATCTTACAG
TGGAGGATGAAGAAGATGGCCATCGGATCCTCAACTCACTCTTCGAGCGTCTTAATG
AAGGACATTCAAAGCCAATTCGAGCAGCTGAAACTGCGGTGGGAGTCTTATCCCAA
TTTGGTCAAGAGCACCGATTATCACCAGAAGAGGGAGACAATTAGACTGGTCACGG
AAGAACTTTATCTTTTAAGTAAAAGAATTGATGATAACATATTGTTCCACAAAACAG
TAATAGCTAACAGCTCCATAATAGCTGACATGGTTGTATCATTATCATTATTAGAAA
CATTGTATGAAATGAAGGATGTGGTTGAAGTGTACAGCAGGCAGTGCTTGTGAATTT
AAAATAAAAATCCTCTTGTTACTACT

A particularly preferred LAIVB for co-compositions, which also serves as a LAIV backbone for chimeric embodiments backbone is 8308B-DelNS1, is disclosed in PCT/CN2018/115938 (published as WO 2020/097923), incorporated herein by reference. Briefly, 8308B- DelNS1 is a Live attenuated influenza virus (LAW) B “LAIVB”) from Victoria lineage is provided, with a deletion of the viral virulence element, the NS1 (non-structural protein 1), 8308B-DelNS1. The 8308B-DelNS1 [preferably, additionally includes adaptive nucleotide mutations (AM) in segments 3 and 5-7 as follows: PA(T210C), NA T(1424C), NP(C182T) and M (A281G). The 8308B-DelNS1 with adaptive mutations is referred to herein as AM/8308B-DelNS1. The AM/8308B DelNS1, is not able to replicate in interferon-competent cells, for example, A549 cells, and preferably replicates at low temperatures such as temperatures below 37° C., more preferably between 30 and 33° C. and most preferably, at about 33° C. In a particularly preferred embodiment, the disclosed LAIVB is characterized in that it replicated poorly in MDCK cells at 37° C., when compared to its replication at 33° C. in the MDCK cells. In a particularly preferred embodiment, the mutated LAIVB/DelLNS1 is able to replicate at levels comparable to wild type influenza virus of the same strain, in a vaccine producing system for example, eggs, or MDCK cells. For example, the 8308B-DelNS1 is able to replicate at levels >10⁷ plague forming units (pfu/ml) for example, between 10⁷-10⁸ pfu/ml. Similar mutations as disclosed herein for the 8308B-DelNS1 or AM/8308B-DelNS1 backbone can be introduced into the respective segments for other influenza B virus; the segment sequences which are publicly disclosed. For example, the GenBank accession number for various influenza B virus segment 7 nucleotide sequence are: DQ792908.1 (Influenza B virus (B/Lee/40) cold-adapted M1 protein (M1) and BM2 protein (BM2) genes, complete cds); M20175.1 (Influenza B/Ann Arbor/1/66 (cold-adapted) membrane protein M1 (seg 7) RNA, complete cds); CY018758.1 (Influenza B virus (B/Victoria/02/1987) segment 7, complete sequence); CY018686.1 (Influenza B virus (B/Hong Kong/1434/2002) segment 7, complete sequence. The 8308B-DelNS1 influenza B virus, designated DeINS1-B8038HK, has been deposited with the American Type Culture Collection, and has ATCC Deposit No. PTA-125209. 8308B-DelNS1, DelNS1-8308B and DeINS1-B8038HK are used herein, interchangeably.

The 8308B-DelNS 1 can be used to express other coronavirus antigens or antigen derived from other clinically significant respiratory viral agents.

Useful LAIVA for the co-compositions disclosed herein are disclosed in U.S. Publication No. 2019/012585, incorporated herein by reference. LAIVA is preferably based on an H1N1 influenza virus genome that includes a deletion of a virulence factor activity, a first set of one or more mutation(s) that confers replication at 37° C. in the absence of the virulence factor activity, and a second set of one or more mutation(s) that confers replication at a temperature below 35° C. The deletion of virulence factor activity can include a deletion of at least part of a virulence factor gene. Such a deletion can be a deletion of at least part of an NS1 gene extending beyond nucleotides 57 to 528 of an NS1 segment of the mutated virus. The first set of one or more point mutation(s) that confer replicative competence, which can lie outside of an M region of the mutated H1N1 influenza virus (for example, a G346A mutation in the H1N1 influenza virus genome). The second set of one or more mutation(s) can include one or more point mutation(s), such as a T261G or an A310G mutation in the H1N1. A particularly preferred LAIVA is disclosed in U.S. Publication No. 2019/012585, developed from such a 2009 H1N1 strain, A/California/04/09 from which an extended portion or all of the NS1 encoding intron (e.g. extending beyond the segment encompassed by nucleotides 57 to 528) has been deleted. U.S. Publication No. 2019/012585 discloses a DelNS1-A strain (therein the cold adapted (CA)4-DelNS1) of such an influenza virus A, which includes a G345A mutation within the NP region, which enables efficient replication of the DelNS1-A virus in cell culture and in eggs. CA4-DelNS1 includes two adaptive substitutions, T261G (L79V) and A310G (E95G), in the NEP coding region of NS segment.

(iii) CoV2Ag

Despite similarities between SARS-CoV and SARS-CoV-2, there is genetic variation between the two and it is not obvious if epitopes that elicit an immune response against SARS -CoV will be effective against Sars-CoV-2.

A preferred CoV2Ag is the receptor binding domain (RBD) of SARS-CoV-2, resulting in the chimeric virus denoted herein as DelNS1-A/B Sars-CoV-2-RBD. The A/B-Strain-DelNS1-SARS-CoV-2-RBD LAIV platform involves distinguishing features in which the key virulent element, NS1, is knocked out, but A/B-Strain-DelNS1-Sars-CoV-2-RBD LAIV can still replicate in vaccine production systems (eggs or MDCK cells). When the receptor-binding domain (RBD) of SARS-CoV-2 is inserted into the NS1 site of viral genome, RBD is stably expressed from cells infected with A/B-Strain-DelNS1-Sars-CoV-2-RBD LAIV.

Use of RBD as antigen minimizes potential antibody-dependent enhancement pathology caused by using full-length spike protein or whole virus as shown in SARS coronavirus. Thus, in some preferred embodiments, the antigen is not full length spike protein of Sars-CoV-2. The RBD can be further optimized to cover more than one strain of coronavirus to prevent future emerging coronavirus. A/B-Strain-DelNS1-Sars-CoV-2-RBD chimeric viruses can induce both neutralizing antibodies and T cell immunities. Various vaccine seeds with different combination of HA and NA of influenza surface proteins can be generated. A/B-Strain-DelNS1-Sars-CoV-2-RBD chimeric viruses can be produced by engineering an influenza virus with a deleted NS1 segment to express RBD. The resulting chimeric viruses include, but are not limited to CA04-DelNS1-Sars-CoV-2-RBD ; HK68-DelNS1-Sars-CoV-2-RBD; 4801-DelNS1-Sars-CoV-2-RBD, H1N1(2019)-DelNS1-Sars-CoV-2-RBD, 8308B-DelNS1-Sars-CoV-2-RBD. These are all A/B-Strain-DelNS1-Sars-CoV-2-CoV2Ag, in which the CoV2Ag portion is RBD. The disclosed chimeric viruses can be used to prepare a live attenuated vaccine that includes the A/B-Strain-DelNS1-Sars-CoV-2-CoV2Ag, as disclosed under formulations, below.

The full genome sequences of CA04-DelNS1-nCoV-RBD deposited into GenBank and GenBank accession nos are MT227009-MT227016. CA04-DelNS1-nCoV-RBD vaccine seed was prepared as disclosed herein was deposited on Apr. 7, 2020 in the American Type Culture Collection (ATCC), 10801 University Boulevard, Manassas, VA 20110 USA, and given Patent Deposit Number PTA-126682.The disclosed chimeric viruses can be used to prepare a live attenuated vaccine that includes the DelNS1-Sars-CoV-2-CoV2AgCoV2Ag as disclosed under formulations, below.

B. Adjuvants

The disclosed LAIV can be administered in conjunction with other immunoregulatory agents, including adjuvants. Useful adjuvants but are not limited to, one or more set forth below:

Mineral Containing Adjuvant Compositions include mineral salts, such as aluminum salts and calcium salts. Exemplary mineral salts include hydroxides (e.g., oxyhydroxides), phosphates (e.g., hydroxyphosphates, orthophosphates), sulfates, and the like or mixtures of different mineral compounds (e.g., a mixture of a phosphate and a hydroxide adjuvant, optionally with an excess of the phosphate), with the compounds taking any suitable form (e.g., gel, crystalline, amorphous, and the like), and with adsorption to the salt(s) being preferred. The mineral containing compositions can also be formulated as a particle of metal salt (WO/0023105). Aluminum salts can be included in compositions of the invention such that the dose of Al³⁺ is between 0.2 and 1.0 mg per dose.

Oil-Emulsion Adjuvants suitable for use as adjuvants in the invention can include squalene-water emulsions, such as MF59 (5% Squalene, 0.5% Tween 80, and 0.5% Span 85, formulated into submicron particles using a microfluidizer). See, e.g., WO90/14837, Podda, Vaccine 19: 2673-2680, 2001. Additional adjuvants for use in the compositions are submicron oil-in-water emulsions. Examples of submicron oil-in-water emulsions for use herein include squalene/water emulsions optionally containing varying amounts of MTP-PE, such as a submicron oil-in-water emulsion containing 4-5% w/v squalene, 0.25-1.0% w/v Tween 80 (polyoxyelthylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-s- -n-glycero-3-huydroxyphosphophoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entirety. MF59 can contain 4-5% w/v Squalene (e.g., 4.3%), 0.25-0.5% w/v Tween 80, and 0.5% w/v Span 85 and optionally contains various amounts of MTP-PE, formulated into submicron particles using a microfluidizer such as Model 110Y microfluidizer (Microfluidics, Newton, Mass.). For example, MTP-PE can be present in an amount of about 0-500 μg/dose, or 0-250 μg/dose, or 0-100 μg/dose. Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) can also be used as adjuvants in the invention.

Saponin Adjuvant Formulations can also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations can include purified formulations, such as QS21, as well as lipid formulations, such as Immunostimulating Complexes (ISCOMs; see below). Saponin compositions have been purified using High Performance Thin Layer Chromatography (HPLC) and Reversed Phase High Performance Liquid Chromatography (RP-HPLC). Specific purified fractions using these techniques have been identified, including QS7, QS17, QS18, QS21, QH-A, QH-B and QH-C. A method of production of QS21 is disclosed in U.S. Pat. No. 5,057,540. Saponin formulations can also comprise a sterol, such as cholesterol (see WO96/33739). Combinations of saponins and cholesterols can be used to form unique particles called ISCOMs. ISCOMs typically also include a phospholipid such as phosphatidylethanolamine or phosphatidylcholine. Any known saponin can be used in ISCOMs. For example, an ISCOM can include one or more of Quil A, QHA and QHC. ISCOMs are described in EP0109942, WO96/11711, and WO96/33739. Optionally, the ISCOMS can be devoid of additional detergent. See WO00/07621. A description of the development of saponin based adjuvants can be found at Barr, et al., “ISCOMs and other saponin based adjuvants”, Advanced Drug Delivery Reviews 32: 247-27, 1998. See also Sjolander, et al., “Uptake and adjuvant activity of orally delivered saponin and ISCOM vaccines”, Advanced Drug Delivery Reviews 32: 321-338, 1998.

Virosomes and Virus-Like Particles (VLPs) can also be used as adjuvants. These structures generally contain one or more proteins from a virus optionally combined or formulated with a phospholipid. They are generally non-pathogenic, non-replicating and generally do not contain any of the native viral genome. The viral proteins can be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and- Mouth Disease virus, Retrovirus, Norwalk virus, human Papilloma virus, HIV, RNA-phages, QB-phage (such as coat proteins), GA-phage, fr-phage, AP205 phage, and Ty (such as retrotransposon Ty protein pl).

Bacterial or Microbial Derivatives useful as adjuvants include: (i) Non-Toxic Derivatives of Enterobacterial Lipopolysaccharide (LPS); (ii) lipid derivatives, (iii) immunostimulatory oligonucleotides and ADP-Ribosylating Toxins and Detoxified Derivatives Thereof, (iv) ADP-Ribosylating Toxins and Detoxified Derivatives Thereof. Examples of Non-Toxic Derivatives of LPS Monophosphoryl lipid A (MPL) and 3-O-deacylated MPL (3 dMPL). 3 dMPL is a mixture of 3 De-O-acylated monophosphoryl lipid A with 4, 5 or 6 acylated chains. An example of a “small particle” form of 3 De-O-acylated monophosphoryl lipid A is disclosed in EP 0 689 454. Such “small particles” of 3 dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g., RC-529 (Johnson et al., Bioorg Med Chem Lett, 9: 2273-2278, 1999). Examples of lipid A derivatives can include derivatives of lipid A from Escherichia coli such as OM-174. OM-174 is described for example in Meraldi et al., Vaccine 21: 2485-2491, 2003; and Pajak, et al., Vaccine 21: 836-842, 2003. Examples of immunostimulatory oligonucleotides nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine can be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research 31: 2393-2400, 2003; WO02/26757 and WO99/62923 for examples of analog substitutions. The adjuvant effect of CpG oligonucleotides is further discussed in Krieg, Nature Medicine (2003) 9(7): 831-835; McCluskie, et al., FEMS Immunology and Medical Microbiology (2002) 32:179-185; WO98/40100; U.S. Pat. Nos. 6,207,646; 6,239,116 and 6,429,199. The CpG sequence can be directed to Toll-like receptor (TLR9), such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence can be specific for inducing a Thl immune response, such as a CpG-A ODN, or it can be more specific for inducing a B cell response, such a CpG-B ODN. CpG-A and CpG-B ODNs are discussed in Blackwell, et al., J. Immunol. 170: 4061-4068, 2003; Krieg, TRENDS in Immunology 23: 64-65, 2002, and WO01/95935. In some aspects, the CpG oligonucleotide can be constructed so that the 5′ end is accessible for receptor recognition. Optionally, two CpG oligonucleotide sequences can be attached at their 3′ ends to form “immunomers”. See, for example, Kandimalla, et al., BBRC 306: 948-95, 2003; Kandimalla, et al., Biochemical Society Transactions 31: 664-658, 2003; Bhagat et al., “BBRC 300: 853-861, 2003, and WO03/035836. Bacterial ADP-ribosylating toxins and detoxified derivatives thereof can be used as adjuvants in the invention. For example, the toxin can be derived from E. coli (i.e., E. coli heat labile enterotoxin (LT)), cholera (CT), or pertussis (PTX). The use of detoxified ADP-ribosylating toxins as mucosal adjuvants is described in WO95/17211 and as parenteral adjuvants in WO98/42375. In some aspects, the adjuvant can be a detoxified LT mutant such as LT-K63, LT-R72, and LTR192G. The use of ADP-ribosylating toxins and detoxified derivatives thereof, particularly LT-K63 and LT-R72, as adjuvants can be found in the following references, each of which is specifically incorporated by reference herein in their entirety: Beignon, et al., Infection and Immunity 70: 3012-3019, 2002; Pizza, et al., Vaccine 19: 2534-2541, 2001; Pizza, et al., Int. J. Med. Microbiol 290: 455-461, 2003; Scharton-Kersten et al., Infection and Immunity 68: 5306-5313, 2000; Ryan et al., Infection and Immunity 67: 6270-6280, 2003; Partidos et al., Immunol. Lett. 67: 09-216, 1999; Peppoloni et al., Vaccines 2: 285-293, 2003; and Pine et al., J. Control Release 85: 263-270, 2002.

Bioadhesives and mucoadhesives can also be used as adjuvants in the invention. Suitable bioadhesives can include esterified hyaluronic acid microspheres (Singh et al., J. Cont. Rel. 70:267-276, 2001) or mucoadhesives such as cross-linked derivatives of poly(acrylic acid), polyvinyl alcohol, polyvinyl pyrollidone, polysaccharides and carboxymethylcellulose. Chitosan and derivatives thereof can also be used as adjuvants in the invention disclosed for example in WO99/27960.

Adjuvant Microparticles: Microparticles can also be used as adjuvants. Microparticles (i.e., a particle of about 100 nm to about 150 μm in diameter, or 200 nm to about 30 μm in diameter, or about 500 nm to about 10 μm in diameter) formed from materials that are biodegradable and/or non-toxic (e.g., a poly(alpha-hydroxy acid), a polyhydroxybutyric acid, a polyorthoester, a polyanhydride, a polycaprolactone, and the like), with poly(lactide-co-glycolide) are envisioned, optionally treated to have a negatively-charged surface (e.g., with SDS) or a positively-charged surface (e.g., with a cationic detergent, such as CTAB).

Examples of liposome formulations suitable for use as adjuvants are described in U.S. Pat. Nos. 6,090,406, 5,916,588, and EP 0 626 169.

Additional adjuvants include polyoxyethylene ethers and polyoxyethylene esters. WO99/52549. Such formulations can further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152). In some aspects, polyoxyethylene ethers can include: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, or polyoxyethylene-23-lauryl ether.

PCPP formulations for use as adjuvants are described, for example, in Andrianov et al., Biomaterials 19: 109-115, 1998.1998. Examples of muramyl peptides suitable for use as adjuvants in the invention can include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-s- -n-glycero-3-hydroxyphosphoryloxy)-ethylamine MTP-PE). Examples of imidazoquinolone compounds suitable for use as adjuvants in the invention can include Imiquimod and its homologues, described further in Stanley, “Imiquimod and the imidazoquinolones: mechanism of action and therapeutic potential” Clin Exp Dermatol 27: 571-577, 2002 and Jones, “Resiquimod 3M”, Curr Opin Investig Drugs 4: 214-218, 2003. Human immunomodulators suitable for use as adjuvants in the invention can include cytokines, such as interleukins (e.g., IL-1, IL-2, IL-4, IL-5, IL-6, IL-7, IL-12, and the like), interferons (e.g., interferon-gamma), macrophage colony stimulating factor, and tumor necrosis factor.

Adjuvant Combinations: The adjuvants are used in come preferred embodiments as combinations. For example, adjuvant compositions can include: a saponin and an oil-in-water emulsion (WO99/11241); a saponin (e.g., QS21)+a non-toxic LPS derivative (e.g., 3 dMPL) (see WO94/00153); a saponin (e.g., QS21)+a non-toxic LPS derivative (e.g., 3 dMPL)+a cholesterol; a saponin (e.g., QS21)+3 dMPL+IL-12 (optionally+a sterol) (WO98/57659); combinations of 3 dMPL with, for example, QS21 and/or oil-in-water emulsions (See European patent applications 0835318, 0735898 and 0761231); SAF, containing 10% Squalane, 0.4% Tween 80, 5% pluronic-block polymer L121, and thr-MDP, either microfluidized into a submicron emulsion or vortexed to generate a larger particle size emulsion. Ribi adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox); and one or more mineral salts (such as an aluminum salt)+a non-toxic derivative of LPS (such as 3 dPML).

Aluminum salts and MF59 are examples of adjuvants for use with injectable influenza vaccines. Bacterial toxins and bioadhesives are examples of adjuvants for use with mucosally-delivered vaccines, such as nasal vaccines. All adjuvants noted above and others as generally known in the art to one of ordinary skill can be formulated for intranasal administration using techniques well known in the art.

C. Formulations and Carriers

The composition of the invention can be formulated in pharmaceutical compositions. These compositions can comprise, in addition to one or more of the DelNS1-A/B-Sars-CoV-2-CoV2Ag, a pharmaceutically acceptable excipient, carrier, buffer, stabilizer, or other materials well known to those skilled in the art. Such materials should typically be non-toxic and should not typically interfere with the efficacy of the active ingredient. The precise nature of the carrier or other material can depend on the route of administration, e.g., oral, intravenous, cutaneous or subcutaneous, nasal, intramuscular, or intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose, or other saccharide solution or glycols such as ethylene glycol, propylene glycol, or polyethylene glycol can be included. The term “carrier” refers to a diluent, adjuvant, excipient, or vehicle with which the pharmaceutical composition (e.g., immunogenic or vaccine formulation) is administered.

Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, ethanol and the like. Examples of suitable pharmaceutical carriers are described in “Remington's Pharmaceutical Sciences” by E. W. Martin. The formulation should be selected according to the mode of administration.

For intravenous, cutaneous, or subcutaneous injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants, and/or other additives can be included, as required.

Administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in the latest edition of Remington's Pharmaceutical Science, Mack Publishing Company, Easton, Pa. (“Remington's”).

### III. Methods of Making

A protocol to engineer A/B-DelNS1-Sars-CoV-2-CoV2Ag chimeric virus is provided for in the examples section of Published Application No. 20190125858, incorporated herein by reference. The protocol includes (a) generating an influenza virus for example the California(CA)/04/09 or the B/Hong Kong/8038/2011 strain with the coding region of the NS1 gene removed from its genome. The coding region of the NS1 gene can be removed using methods known in the art. Methods to introduce targeted mutations into a genome or, in the context of virology, into a virus are subsumed under the term reverse genetics (RG) and are disclosed for example, in Hoffmann et al., Proc Natl Acad Sci USA, 97(11):6108-13 Zheng et al., J. Virol. 89(20): 10273-85 and Dauber, et al., J. Virol., 78(4):1865-1872 (2004), the materials and method of which are incorporated herein by reference. The method of generating influenza virus with the NS1 coding region deleted, as disclosed in Published Application No. 20190125858 is generalized and summarized herein. The method of generating influenza B virus with the NS1 coding region deleted, as disclosed PCT/CN2018/115,938 is generalized and summarized herein.

A. Generating Live Attenuated Influenza Virus (LAIV) with the Coding Region of the NS1 Gene Removed

The LAIVA can be constructed in some preferred embodiments as disclosed herein in the

Examples, with the methods disclosed herein exemplifying CA04-DelNS1. Briefly, an NS1 deletion Plasmid is constructed. Construction of NS1 Deletion Plasmid: A suitable viral strain, for example, 2009 H1N1 A/California/04/09 (CA04) can be used as backbone to construct the DelNS1 vaccines strain. Plasmid without NS1 expression can be constructed by inverse PCR with primers as follows: CA04-DelNS1-529F: GACATACTTATGAGGATGTC (SEQ ID NO:29); CA04-DelNS1-56F: CTGAAAGCTTGACATGGTGTTG (SEQ ID NO:30). These primers can be used to construct CA4-DelNS1 virus from a California(CA)/04/09 strain through reverse genetic procedures that delete an intron at 56-529.

Primers 5′-GACATACTGTGAGGATGTCAAAAATG-3′ (NS-529F) (SEQ ID NO:31) and 5′-CTGAAAGCTTGACACAGTGTTTGG-3′ (NS-56R) (SEQ ID NO:32) can be used to construct A/WSN/33-DelNS1 and A/PR/8/34-DELNS1.

The NS1 deletion plasmid can be constructed according to the protocol described in a previous report (Garcia-Sastre, J. Virology 252:324-330, 1998); Zheng, et al., J Virol 89:10273-10285 (2015). In brief, inverse PCR is carried out to delete the intron of the NS gene inserted into the pHW2000 vector and the plasmid phosphorylated and self-ligated. For point mutations, commercially available kits can be used, for example, the QuikChange II site-directed mutagenesis kit (Stratagene).

The LAIVB can be constructed as disclosed in PCT/CN2018/115,938, with the methods disclosed therein exemplifying DelNS1-B8038. Briefly, the protocol includes (a) generating influenza B virus with the coding region of the NS1 gene removed from its genome, by transfecting the eight plasmids, preferably pHW2000 plasmids, containing the genome of DelNS1 influenza B virus including a plasmid to express NS1 protein, into one or more vaccine producing cells with (b) rescuing LAVIB/DelNS1 virus and (c) passaging rescued virus into one or more vaccine producing cells at 33° C. and 37° C. respectively until viral titer is stabilized, for example, when the virus titer remains unchanged for three consecutive passages, to obtain AM/LAVIB/DelNS1 and (c) analyzing for the presence of desired mutations.

In a preferred embodiment, the virus in step b is cultured in a mixture of two or more cells, for example, 293T/MDCK cells. The disclosed methods for making LAIVB result in a deletion of the viral virulence element, the NS1 protein and an adaptive mutation which allows growth of the mutated strain in vaccine producing systems such as eggs and MDCK cells The preferred adaptive mutations are PA(T210C), NA T(1424C), NP(C182T) and M (A281G) mutations. The disclosed methods preferably include reverse genetics. Plasmids containing the deleted NS1 segment (DelNS1) and the other seven genome segments derived from an influenza B virus strain are transfected into 293T/MDCK cells mixture. Preferred virus strains include B/Yamagata and BNictoria Rescued virus, passaged in MDCK cells until virus titer is stabilized, until high levels of viral titer are obtained and remain unchanged for at least three consecutive passages. The virus is sequenced to determine adaptive mutations.

The coding region of the NS1 gene can be removed using methods known in the art. Methods to introduce targeted mutations into a genome or, in the context of virology, into a virus are subsumed under the term reverse genetics (RG) and are disclosed for example, in Hoffmann et al., Proc Natl Acad Sci USA, 97(11):6108-13 Zheng et al., J. Virol. 89(20): 10273-85 and Dauber, et al., J. Virol., 78(4):1865-1872 (2004), the materials and method of which are incorporated herein by reference. The method of generating influenza virus with the NS1 coding region deleted, as disclosed in Dauber et al. is generalized and summarized herein.

(i) RT-PCR and Construction of Plasmids

The RNeasy kit (Qiagen) can be used according to the manufacturer's protocol to extract viral RNA from a stock of influenza B virus. Eight reverse genetic plasmids pHW- PB2, pHW-PB1, pHW- PA, pHW- HA, pHW-NP, pHW-NA, pHW-M1, and pHW-NS1 (Table 1) are constructed by reverse transcriptase PCR (RT-PCR) amplification of single viral RNA segments and the resulting cDNAs cloned into the plasmid pHW2000. All eight plasmids can be used to generate the wild type influenza B virus. Primers that can be used in the PCR amplification are listed in the following Table 2:

TABLE 2

| | |
|---|---|
| Flub-pb1-lic-S (SEQ ID NO: 9) | CcgaagttgggggggAGCAGAAGCGGAGC |
| Flub-pb1-lic-as (SEQ ID NO: 10) | GGCCGCCGGGTTATTAGTAGAAACACGAGC |
| Flub-pb2-lic-S (SEQ ID NO: 11) | CcgaagttgggggggAGCAGAAGCGGAGC |
| Flub-pb2-lic-as (SEQ ID NO: 12) | GGCCGCCGGGTTATTAGTAGAAACACGAGC |
| Flub-pa-lic-S (SEQ ID NO: 13) | CcgaagttgggggggAGCAGAAGCGGTGC |
| Flub-pa-lic-as (SEQ ID NO: 14) | GGCCGCCGGGTTATTAGTAGAAACACGTGC |
| Flub-ha-lic-S (SEQ ID NO: 15) | CcgaagttgggggggAGCAGAAGCAGAGC |
| Flub-ha-lic-as (SEQ ID NO: 16) | GGCCGCCGGGTTATTAGTAGTAACAAGAGC |
| Flub-na-lic-S (SEQ ID NO: 17) | CcgaagttgggggggAGCAGAAGCAGAGC |
| Flub-na-lic-as (SEQ ID NO: 18) | GGCCGCCGGGTTATTAGTAGTAACAAGAGC |
| Flub-np-lic-S (SEQ ID NO: 19) | CcgaagttgggggggAGCAGAAGCACAGC |
| Flub-np-lic-as (SEQ ID NO: 20) | GGCCGCCGGGTTATTAGTAGAAACAACAGC |
| Flub-m-lic-S (SEQ ID NO: 21) | CcgaagttgggggggAGCAGAAGCACGCACTT |
| Flub-m-lic-as (SEQ ID NO: 22) | GGCCGCCGGGTTATTAGTAGAAACAACGCACTT |
| Flub-ns-lic-S (SEQ ID NO: 23) | CcgaagttgggggggAGCAGAAGCAGAGGATT |
| Flub-ns-lic-as (SEQ ID NO: 24) | GGCCGCCGGGTTATTAGTAGTAACAAGAGGATT |
| Flub-delns1-F (SEQ ID NO: 25) | CTCAAT TT GTGTTGTGGTC ATG |
| Flub-delns1-R (SEQ ID NO: 26) | TGGAGGATGAAGAAGATGGCCATCGGATCCTC |

The coding region of the influenza B virus NS 1 protein overlaps in part with the reading frame for the NEP/NS2 protein that is expressed from a spliced transcript of the viral NS gene segment. For the generation of NS1-deficient influenza B virus, a derivative of the NS reverse genetic plasmid termed pHW-Lee-ΔNS1-B is prepared, in which the sequences specifying the NS1 protein were deleted, while all NEP/NS2 coding sequences and the terminal noncoding regions were maintained. For example, for generation of the pHW- DelNS1-B plasmid, polyadenylated RNA is extracted from influenza B virus-infected MDCK cells by using the Oligotex direct mRNA Midi/Maxi Kit (Qiagen) and NS segment-specific primers used for RT-PCR amplification Amplified NEP/NS2 fragment is purified with the QiaEx II gel extraction kit (Qiagen), digested with BsmBI, and cloned into pHW2000.

These plasmids facilitate bidirectional transcription of negative-sense viral RNAs and positive-sense mRNAs since the cloned viral cDNAs are flanked upstream by a human RNA polymerase I promoter and downstream by an RNA polymerase II-specific promoter. In brief, the viral RNAs are first reverse transcribed with Moloney murine leukemia virus reverse transcriptase (Promega) by using a universal nine-nucleotide primer (UNI-9) that is complementary to the conserved 3′ ends of all eight viral RNA segments. The RT reaction is performed for 60 min at 37° C., followed by 15 min at 70° C. Subsequently, single gene segments are amplified by PCR by using the Pfu Turbo Polymerase (Roche Diagnostics) and segment-specific primers carrying BsmBI (PB1, PB2, PA, NA, M, and NS), BspMI (NP), or AarI (HA) restriction site sequences at their 5′ ends. pHW-NS-XhoI is a derivative of pHW-NS that is constructed with the QuikChange mutagenesis kit (Stratagene) by introducing a novel XhoI recognition site at nucleotides 262 to 267 of the viral NS segment. The NS segment of the transfectant virus is engineered to carry an engineered genetic tag site such that the corresponding cDNA is susceptible to cleavage by the restriction endonuclease XhoI, thereby verifying the recombinant nature of the isolate.

(ii) Transfection-Mediated Recovery of Recombinant Influenza B Virus.

To generate the recombinant influenza B wild-type virus the eight plasmids pHW-PB2, pHW-PB1, pHW-PA, pHW-HA, pHW-NP, pHW-NA, pHW-M, and pHW-NS-XhoI (0.5 μg each) are transfected into 10⁶ 293T cells in suspension with the Lipofectamine 2000 reagent (Invitrogen). At 72 h after transfection, the supernatant of transfected cells is inoculated into the allantoic cavities of 11-day-old chicken eggs to grow stocks of recombinant virus. Supernatants of transfected cells harboring mutant plasmid can be passaged into 6-day-old chicken eggs. The recovery of recombinant influenza B viruses can be verified by gel electrophoretic analyses of RT-PCR products representing the viral NS segments.

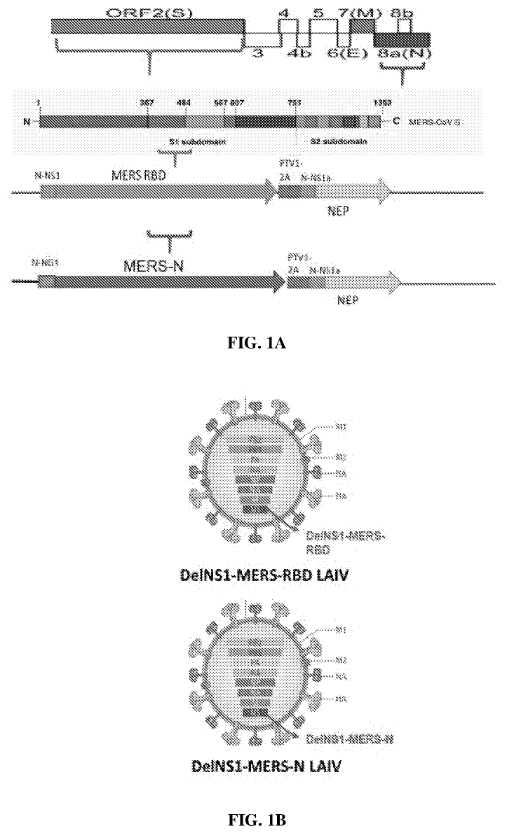

The plasmids are preferably transfected into a mixture of cells, for example, 293T/MDCK cells to grow stocks of recombinant virus. FIG. 1A. The DelNS1 virus can be rescued by essentially the same procedure, except that pHW-NS-XhoI is replaced by pHW-ΔNS1-B and 0.5 μg of an expression vector for NS1, for example, pcDNA-NS1 added to the transfection mix. To construct plasmid pcDNA-NS1-B, the NS1 cDNA can be PCR amplified with pHW-Lee-NS as a template and cloned between the HindIII/XhoI sites of pcDNA3

The influenza B virus completely lacking the NS1 ORF disclosed in Dauber, et al. J. Virol., 78:1865-1872 (2004) does not replicate efficiently in Vero cells (titres of 1.7-2.5*10² FFU/ml using an moi of 0.1 and no detectable titres at moi of 0.001, respectively. An influenza B NS1 deletion mutant consisting of the amino-terminal 16 aa is also highly attenuated in replication with maximum titres of approx. 10⁴ FFU/ml. (Hai et. al; Journal of Virology; November 2008, p. 10580-90. These types of influenza viruses disclosed in this paragraph, preferably, do not serve as backbone for the chimeric viruses disclosed herein.

Construction of the pHW2000-Sars-CoV-2-RBD-NEP Plasmid

The plasmid including the Sars-CoV-2-RBD can be prepared adapting the method disclosed for the pHW2000-MERS-RBD-NEP Plasmid in U.S. Published application No. 2019/0125858.

Briefly, to generate recombinant NS1-deleted influenza virus expressing Sars-CoV-2 receptor binding domain (RBD), a pHW2000-Sars-CoV-2-RBD-NEP plasmid can be constructed. It has an open reading frame which is composed of B8038 N terminal of NS1, Sars-CoV-2 RBD domain, PTV1-2A cleavage site, DelNS1-B8038 NEP with the mutated N terminal NS1 sequence.

The sequence of Sars-CoV-2-RBD-PTV1-2A is amplified by PCR and inserted into the pHW2000- DelNS1-B8038, which contains only B8038 NEP open reading frame, by ligation independent cloning using exonuclease III. After transformation, plasmids were extracted from right clones and subsequently sequenced to confirm the sequence.

Rescue of CA-04-DELNS1 Virus

Nine plasmids: pHW2000-CA04-PB2, pHW2000-CA04-PB1, pHW2000-CA04-PA, pHW2000-CA04-NP, pHW2000-CA04-HA, pHW2000-CA04-NA, pHW2000-CA04-M, pHW2000-CA04-DelNS1 and pCX-CA04-NS1 are mixed together in one tube. Each one is present at 1 μg. Transfection with the mixed plasmids was conducted in 80% confluent 293T cells plated in a 6-well plate. During transfection the old medium was replaced with 1 ml Opti-MEM without penicillin and streptomycin. Sixteen hours later the supernatant was discarded and 2 ml of MEM containing 1 μg/ml trypsin was added. Seventy hours after transfection, the supernatant was collected after the cell debris was removed.

Passage of DelNS1 Virus

Two hundred microliter rescued DelNS1 virus can be injected into a 9 to 10-day-old fertilized egg and incubated in the 37° C. incubator for 48 hours. Egg allantoic fluid was collected and HA titer was measured. Blood cells and other debris were removed by centrifugation at 1500 g for 10 minutes. Supernatant was transferred into a Millipore 100K ultra filter and centrifuged at the speed of 3000 g for 10 minutes. PBS was added to the filter to give a volume of 10 ml to wash the concentrated virus, and the suspension was again centrifuged at 3000 g for 10 minutes. Two hundred microliter of the resulting virus preparation is used to inoculate 9 to 10-day-old fertilized eggs and the procedure was repeated until the virus HA titer increased dramatically.

Rescued DelNS1-Sars-CoV-2-CoV2Ag chimeric virus can be cultured in any virus-producing cell until virus titer is stabilized, evidenced for example, when the virus titer remains unchanged for at least three consecutive passages in MDCK cells and eggs. Supernatant from the transfected cells after 72 hours is collected and passaged in MDCK cells.

A preferred cell for passaging is MDCK (Madin-Darby canine kidney) cells. However, the cells used for the cultivation of viruses using a cultivation medium can be cells that can grow in vitro in synthetic media and can be used for the propagation of viruses. These can be for example BSC-1 cells, LLC-MK cells, CV-1 cells, CHO cells, COS cells, murine cells, human cells, HeLa cells, 293 cells, VERO cells, MDBK cells, MDOK cells, CRFK cells, RAF cells, TCMK cells, LLC-PK cells, PK15 cells, WI-38 cells, MRC-5 cells, T-FLY cells, BHK cells, SP2/0 cells, NSO, PerC6 (human retina cells), chicken embryo cells or derivatives, embryonated egg cells, embryonated chicken eggs or derivatives thereof.

The cultivation medium used for the production of viruses can be any medium known from prior art that is applicable for virus cultivation. Preferably the medium is a synthetic medium. This can be for example basal media as Modified Eagle's media MEM, minimum essential media MEM, Dulbecco's modified Eagle's media D-MEM, D-MEM-F12 media, William's E media, RPMI media and analogues and derivative thereof. These can also be specialty cell cultivation and virus growth media as VP-SFM, OptiPro™ SFM, AIM V® media, HyQ SFM4 MegaVir™, EX-CELL™ Vero SFM, EPISERF, ProVero, any 293 or CHO media and analogues and derivatives thereof. These media can be supplemented by any additive known from prior art that is applicable for cell and virus cultivation as for example animal sera and fractions or analogues thereof, amino acids, growth factors, hormones, buffers, trace elements, trypsin, sodium pyruvate, vitamins, L-glutamine and biological buffers. Preferable medium is OptiPRO™ SFM supplemented with L-glutamine and trypsin.

Thus, disclosed method includes culturing the virus in for an effective amount of time to obtain a stable viral titer. In preferred embodiment, the rescued virus is passaged in a virus-producing cell, for example, MDCK cells for a period of time until viral titre remains unchanged for 3 consecutive passaged. This culture period can range from 10-50 passages, preferably, for over 20 passages at 33° C. The time and conditions of culture result in adaptive mutations, which allows replication of the LAIVB in vaccine producing systems such as eggs or MDCK. The examples DelNS1-Sars-CoV-2-RBD can replicate in the vaccine producing cell line, MDCK cells, for the viral strain tested.

(iii) Construction of the Sars-CoV-2-CoV2Ag/Influenza HA/NA Plasmid

A plasmid including Sars-CoV-2 antigen or NA/HA of an influenza strain of choice can be prepared as exemplified here for Sars-CoV-2-RBD. The RBD can simply be replaced with the Sars-CoV-2 antigen of choice or the NA/HA from different strain (than the backbone LAIV being used) of influenza virus.

Construction of the pHW2000-Sars-CoV-2-RBD-NEP Plasmid

The plasmid including the Sars-CoV-2-RBD can be prepared adapting the method disclosed for the pHW2000-MERS-RBD-NEP Plasmid in U.S. Published application No. 2019/0125858.

Briefly, to generate recombinant NS1-deleted influenza virus expressing Sars-CoV-2 receptor binding domain (RBD), a pHW2000-Sars-CoV-2-RBD-NEP plasmid can be constructed. It has an open reading frame which is composed of CA04 N terminal of NS1, Sars-CoV-2 RBD domain, PTV1-2A cleavage site, CA04 NEP with the mutated N terminal NS1 sequence.

The sequence of Sars-CoV-2-RBD-PTV1-2A is amplified by PCR and inserted into the pHW2000-CA04-DelNS1, which contains only CA04 NEP open reading frame, by ligation independent cloning using exonuclease III. After transformation, plasmids were extracted from right clones and subsequently sequenced to confirm the sequence.

(iv) Rescue of the DELNS1-Sars-CoV-2CoV2Ag Chimeric Virus

Rescue of the A-Strain-DELNS1-Sars-CoV-2CoV2Ag chimeric virus is exemplified herein using the CA04-delNS1-RBD Virus. These methods are applicable to rescue of chimeric virus using other LAIV backbone, for example, HK68-DELNS1-Sars-CoV-2-RBD; 4801-DELNS1 -Sars-CoV-2-RBD and H1N1(2019)-DELNS1-Sars-CoV-2-RBD.

Rescue of the CA04-delNS1-RBD Virus

Nine plasmids: pHW2000-CA04-PB2, pHW2000-CA04-PB1, pHW2000-CA04-PA, pHW2000-CA04-NP, pHW2000-CA04-HA, pHW2000-CA04-NA, pHW2000-CA04-M, pHW2000-Sars-CoV-2-RBD-NEP and pCX-CA04-NS1, each with 1μg, are mixed and used to transfect 80% confluent 293T cells in a 6-well plate. During transfection the old medium is replaced with 1 ml of Opti-MEM without antibiotics. Sixteen hours later the supernatant is discarded and 2 ml of MEM containing 1 ng/ml trypsin added. Seventy hours after transfection, the supernatant was collected after the cell debris is removed. The supernatant is injected into 9 to 10-day-old fertilized eggs and incubated at 37° C. for 48 hours. Egg allantoic fluid is collected, and cleared by centrifugation. The virus is then sequenced and titred by plaque assay in MDCK cells.

Rescue of the B-Strain-DELNS1-Sars-CoV-2-CoV2Ag Chimeric Virus

Rescue of B-Strain-DELNS1-Sars-CoV-2-CoV2Ag chimeric virus is exemplified herein using the delNS1-B8038-RBD Virus. These methods are applicable to rescue of chimeric virus using other LAIVB backbone.

DelNS1-B8038-RBD Virus can be rescued using the following protocol. Nine plasmids: pHW2000-B8038-PB2, pHW2000- B8038-PB1, pHW2000- B8038-PA, pHW2000- B8038-NP, pHW2000- B8038-HA, pHW2000-B8038-NA, pHW2000-B8038-M, pHW2000-Sars-CoV-2-RBD-NEP and pCX-B8038-NS1, each with 1 μg, are mixed and used to transfect 80% confluent 293T cells in a 6-well plate. During transfection the old medium is replaced with 1 ml of Opti-MEM without antibiotics. Sixteen hours later the supernatant is discarded and 2 ml of MEM containing 1 ng/ml trypsin added. Seventy hours after transfection, the supernatant was collected after the cell debris is removed. The supernatant is injected into 9 to 10-day-old fertilized eggs and incubated at 37° C. for 48 hours. Egg allantoic fluid is collected, and cleared by centrifugation. (iii) The virus is then sequenced and tittered by plaque assay in MDCK cells.

(v) Passage and Rescue of DelNS1-B and Chimeric B-Strain-DelNS1-Sars-CoV-2-Cov2Ag Virus Strains

Rescued DelNS1-B and DelNS1-B-Sars-CoV-2-CoV2Ag chimeric virus can be cultured in any virus-producing cell until virus titer is stabilized, evidenced for example, when the virus titer remains unchanged for at least three consecutive passages in MDCK cells and eggs. Supernatant from the transfected cells after 72 hours is collected and passaged in MDCK cells.

A preferred cell for passaging is MDCK (Madin-Darby canine kidney) cells. However, the cells used for the cultivation of viruses using a cultivation medium can be cells that can grow in vitro in synthetic media and can be used for the propagation of viruses. These can be for example BSC-1 cells, LLC-MK cells, CV-1 cells, CHO cells, COS cells, murine cells, human cells, HeLa cells, 293 cells, VERO cells, MDBK cells, MDOK cells, CRFK cells, RAF cells, TCMK cells, LLC-PK cells, PK15 cells, WI-38 cells, MRC-5 cells, T-FLY cells, BHK cells, SP2/0 cells, NSO, PerC6 (human retina cells), chicken embryo cells or derivatives, embryonated egg cells, embryonated chicken eggs or derivatives thereof.

DelNS1-B-Sars-CoV-2-CoV2Ag chimeric virus can also be passaged in eggs using a protocol exemplified as follows. Two hundred microliter rescued DelNS1-B virus can be injected into a 9 to 10-day-old fertilized egg and incubated in the 37° C. incubator for 48 hours. Egg allantoic fluid was collected and HA titer was measured. Blood cells and other debris are removed by centrifugation at 1500 g for 10 minutes. Supernatant is transferred into a Millipore 100K ultra filter and centrifuged at the speed of 3000 g for 10 minutes. PBS was added to the filter to give a volume of 10 ml to wash the concentrated virus, and the suspension was again centrifuged at 3000 g for 10 minutes. Two hundred microliter of the resulting virus preparation is used to inoculate 9 to 10-day-old fertilized eggs and the procedure was repeated until the virus HA titer increased dramatically.

The cultivation medium used for the production of viruses can be any medium known from prior art that is applicable for virus cultivation. Preferably the medium is a synthetic medium. This can be for example basal media as Modified Eagle's media MEM, minimum essential media MEM, Dulbecco's modified Eagle's media D-MEM, D-MEM-F12 media, William's E media, RPMI media and analogues and derivative thereof. These can also be specialty cell cultivation and virus growth media as VP-SFM, OptiPro™ SFM, AIM V® media, HyQ SFM4 MegaVir™, EX-CELL™ Vero SFM, EPISERF, ProVero, any 293 or CHO media and analogues and derivatives thereof. These media can be supplemented by any additive known from prior art that is applicable for cell and virus cultivation as for example animal sera and fractions or analogues thereof, amino acids, growth factors, hormones, buffers, trace elements, trypsin, sodium pyruvate, vitamins, L-glutamine and biological buffers. Preferable medium is OptiPRO™ SFM supplemented with L-glutamine and trypsin.

Thus, disclosed method includes culturing the virus in for an effective amount of time to obtain a stable viral titer. In preferred embodiment, the rescued virus is passaged in a virus-producing cell, for example, MDCK cells for a period of time until viral titre remains unchanged for 3 consecutive passaged. This culture period can range from 10-50 passages, preferably, for over 20 passages at 33° C. The time and conditions of culture result in adaptive mutations, which allows replication of the LAIVB in vaccine producing systems such as eggs or MDCK. The examples DelNS1-Sars-CoV-2-RBD can replicate in the vaccine producing cell line, MDCK cells, for the viral strain tested.

Other domains (other than NS1 disclosed herein) of the influenza virus such as the HA and NA gene segments can be engineered to express a heterologous gene of interest, using methods known in the art. Additionally, it is also possible to express full-length foreign proteins through an additional gene segment. Thus, the disclosed LAIVA/B could be modified to express the HA/NA gene of interest, from a different influenza strain using similar methods described in Gao et al., J Virol. 2010; 84:8062-71. Influenza virus genomic RNAs possess segment-specific packaging signals that include both noncoding regions (NCRs) and adjacent terminal coding region sequences. Using reverse genetics, the disclosed LAIVA/B can be rescued that contains a modified PB1 gene such that the PB1 packaging sequences were exchanged for those of the neuraminidase (NA)/HA gene segment of choice. To accomplish this, the PB1 open reading frame, in which the terminal packaging signals were inactivated by serial synonymous mutations, was flanked by the NA segment-specific packaging sequences including the NCRs and the coding region packaging signals. Next, the ATGs located on the 3′ end of the NA packaging sequences of the resulting PB1 chimeric segment were mutated to allow for correct translation of the full-length PB1 protein. See Gao et al., J Virol. 2010; 84:8062-71, materials and methods.

### IV. Methods of Use

The disclosed DelNS1-A/B-Sars-CoV-2-CoV2Ag chimeric virus can be used to effectively increase viral titer or elicit an immune response in a subject in need thereof. In some aspects, subjects can include the elderly (e.g., >65 years old), young children (e.g., <5 years old). Methods for improving immune response in children using adjuvanted formulations are disclosed for example in U.S. Publication 2017/0202955. DelNS1-A/B-Sars-CoV-2-CoV2Ag, for example, B8038- DelNS1-SARS-CoV-2-RBD LAIV can induce both neutralizing antibodies and T cell immunities.

Methods for improving immune response in children using adjuvanted formulations are disclosed for example in U.S. Publication 2017/0202955.

The DelNS1-A/B Sars-CoV-2-CoV2Ag chimeric virus stains can generally be administered directly to a mammal in need thereof to increase viral titer in the mammal and elicit an immune response. In some embodiments the subject is a young child, less than 5 years of age. In other embodiments, the subject is a young child, less than two years of age. In the embodiments, the composition is administered intranasally. In other embodiments the subject is elderly, and the subject can be between the ages of 5 and 65.

Viruses are typically administered to a patient in need thereof in a pharmaceutical composition. Pharmaceutical compositions containing virus may be for systemic or local administration. Dosage forms for administration by parenteral (intramuscular (IM), intraperitoneal (IP), intravenous (IV) or subcutaneous injection (SC)), or transmucosal (nasal, vaginal, pulmonary, or rectal) routes of administration can be formulated. In the most preferred embodiments, the immunizing virus is delivered peripherally by intranasally or by intramuscular injection, and the therapeutic virus is delivered by local injection.

Direct delivery can be accomplished by parenteral injection (e.g., subcutaneously, intraperitoneally, intradermal, intravenously, intramuscularly, or to the interstitial space of a tissue), or mucosally, such as by rectal, oral (e.g., tablet, spray), vaginal, topical, transdermal (See e.g., WO99/27961) or transcutaneous (See e.g., WO02/074244 and WO02/064162), inhalation, intranasal (See e.g., WO03/028760), ocular, aural, pulmonary or other mucosal administration. Compositions can also be administered topically by direct transfer to the surface of the skin. Topical administration can be accomplished without utilizing any devices, or by contacting naked skin with the composition utilizing a bandage or a bandage-like device (see, e.g., U.S. Pat. No. 6,348,450). In some aspects, the mode of administration is parenteral, mucosal, or a combination of mucosal and parenteral immunizations. In other aspects, the mode of administration is parenteral, mucosal, or a combination of mucosal and parenteral immunizations in a total of 1-2 vaccinations 1-3 weeks apart. In related aspects, the route of administration includes but is not limited to intranasal delivery.

A. Effective Amounts

Typically the composition is administered in an effective amount to induce an immune response against a one or more Sars-CoV-2 antigens encoded by the chimeric virus. For example, an effective amount of virus generally results in production of antibody and/or activated T cells that kill or limit proliferation of or infection by the Sars-CoV-2.

The composition can typically be used to elicit systemic and/or mucosal immunity, for example to elicit an enhanced systemic and/or mucosal immunity. For example, the immune response can be characterized by the induction of a serum IgG and/or intestinal IgA immune response. Typically, the level of protection against influenza infection can be more than 50%, e.g., 60%, 70%, 80%, 90%, 91%, 92%, 93%, 94%, 95%, 96%, 97%, 98%, 99%, or more. In one aspect, the level of protection can be 100%.

The immune response induced by the invention can be one or both of a TH1 immune response and a TH2 response. The immune response can be an improved or an enhanced or an altered immune response. The immune response can be one or both of a systemic and a mucosal immune response. For example, the immune response can be an enhanced systemic and/or mucosal response. An enhanced systemic and/or mucosal immunity is reflected in an enhanced TH1 and/or TH2 immune response. For example, the enhanced immune response can include an increase in the production of IgG1 and/or IgG2a and/or IgA. In another aspect the mucosal immune response can be a TH2 immune response. For example, the mucosal immune response can include an increase in the production of IgA.

Typically, activated TH2 cells enhance antibody production and are therefore of value in responding to extracellular infections. Activated TH2 cells can typically secrete one or more of IL-4, IL-5, IL-6, and IL-10. A TH2 immune response can also result in the production of IgG1, IgE, IgA, and/or memory B cells for future protection. In general, a TH2 immune response can include one or more of an increase in one or more of the cytokines associated with a TH2 immune response (such as IL-4, IL-5, IL-6 and IL-10), or an increase in the production of IgG1, IgE, IgA and memory B cells. For example, an enhanced TH2 immune response can include an increase in IgG1 production. A TH1 immune response can include one or more of an increase in CTLs, an increase in one or more of the cytokines associated with a TH1 immune response (such as IL-2, IFN-gamma, and TNF-alpha), an increase in activated macrophages, an increase in NK activity, or an increase in the production of IgG2a. For example, the enhanced TH1 immune response can include an increase in IgG2a production.

The DelNS1-Sars-CoV-2-CoV2Ag chimeric virus strains can be used either alone or in combination with other agents optionally with an immunoregulatory agent capable of eliciting a Th1 and/or Th2 response.

B. Dosages

The precise dosage will vary according to a variety of factors such as subject-dependent variables (e.g., age, immune system health, etc.), and age of the subject being treated. Appropriate dosages can be determined by a person skilled in the art, considering the therapeutic context, age, and general health of the recipient. The selected dosage depends upon the desired therapeutic effect, on the route of administration, and on the duration of the treatment desired. In determining the effective amount of the virus to be administered for the prophylaxis, the physician may evaluate circulating plasma levels of virus, and/or the production of existing antibodies against the antigen(s). Active virus can also be measured in terms of plaque-forming units (PFU). A plaque-forming unit can be defined as areas of cell lysis (CPE) in monolayer cell culture, under overlay conditions, initiated by infection with a single virus particle. Generally, dosage levels of virus between 10² and 10¹² pfu are administered to humans In different embodiments, the dosage range is from 10⁴ to 10¹⁰ pfu, 10⁵ to 10⁹ pfu, 10⁶ to 10⁸ pfu, or any dose within these stated ranges. When more than one vaccine is to be administered (i.e., in combination vaccines), the amount of each vaccine agent can be within their described ranges.

Virus is typically administered in a liquid suspension, in a volume ranging between 10 μl and 100 μl depending on the route of administration. Vaccine volumes commonly practiced range from 0.1 mL to 0.5 mL. Generally, dosage and volume will be lower for local injection as compared to systemic administration or infusion.

The vaccine composition can be administered in a single dose or a multi-dose format. Vaccines can be prepared with adjuvant hours or days prior to administrations, subject to identification of stabilizing buffer(s) and suitable adjuvant composition. Typically, the dose will be 100 μl administered locally in multiple doses, while systemic or regional administration via subcutaneous, intramuscular, intra-organ, intravenous or intranasal administration can be from for example, 10 to 100 μl

### V. Kits

A kit including the disclosed DelNS1-A/B-Sars-CoV-2-CoV2Ag chimeric virus strains are also provided. The kit can include a separate container containing a suitable carrier, diluent or excipient. Additionally, the kit can include instructions for mixing or combining ingredients and/or administration.

Compositions can be in liquid form or can be lyophilized. Suitable containers for the compositions include, for example, bottles, vials, syringes, and test tubes. Containers can be formed from a variety of materials, including glass or plastic. A container can have a sterile access port (for example, the container can be an intravenous solution bag or a vial having a stopper pierceable by a hypodermic injection needle).

The kit can further include a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device(s). The kit can further include a third component comprising an adjuvant.

The kit can also include a package insert containing written instructions for methods of inducing immunity, preventing infections, or for treating infections. The package insert can be an unapproved draft package insert or can be a package insert approved by the Food and Drug Administration (FDA) or other regulatory body.

The invention also provides a delivery device pre-filled with the compositions of the invention.

The compositions are generally formulated as sterile, substantially isotonic and in full compliance with all Good Manufacturing Practice (GMP) regulations of the U.S. Food and Drug Administration.

The disclosed compositions, and methods can be further understood through the following numbered paragraphs.
1. A live attenuated chimeric virus comprising (a) an influenza virus genome, wherein the influenza virus genome comprises a deletion of a virulence factor activity, and optionally, a first set of one or more mutation(s) that confers replication at 37° C. in the absence of the virulence factor activity; and a second set of one or more mutation(s) that confers replication at a temperature below 35° C., and (b) an insertion of one or more genes encoding one or more Sars-CoV-2 antigens (CoV2Ag).
2. The attenuated chimeric virus of paragraph 1, wherein the influenza virus genome is from an influenza virus A subtype H1N1 or H3N2.
3. The attenuated chimeric virus of paragraph 2, wherein the influenza virus genome is from an influenza virus A subtype H1N1 or H3N2 strain selected from the group consisting of CA04 (A/California/04/2009); HK6 8 (strain A/Hong Kong/1/68), 4801 (H3N2 A/HK/4801/2014), H1N1(2019); A/WSN/33 and A/PR/8/34.
4. The attenuated chimeric virus any one of paragraphs 1-3, wherein the deletion of virulence factor activity comprises a deletion of at least part of a virulence factor gene.
5. The chimeric virus of any one of paragraphs 1-4, wherein the deletion comprises a deletion of at least part of Non-Structural Protein 1 (NS1) gene extending beyond nucleotides 57 to 528 of an NS1 segment of the mutated virus.
6. The chimeric virus of any one of paragraphs 1-5, comprising a first set of one or more mutation(s), wherein the first set of one or more mutation(s) comprises a first set of one or more point mutation(s) that confer replicative competence.
7. The chimeric virus any one of paragraphs 1-5, wherein the first set of one or more point mutation(s) lies outside of an M region of the mutated influenza virus.
8. The chimeric virus s of paragraph 3, wherein the influenza virus genome is from the A/California/04/2009 influenza strain, and at least one of the first set of one or more point mutation(s) is a G346A mutation in the viral genome.
9. The chimeric virus of any one of paragraphs 1-8, wherein the virus replicates poorly in MDCK cells at 37° C., when compared to its replication at 33° C. in the MDCK cells.
10. The chimeric virus of any one of paragraphs 1-7, wherein the second set of one or more mutation(s) comprises a second set of one or more point mutation(s).
11. The chimeric virus of paragraph 3, wherein the first set of one or more mutation(s) comprises an A14U substitution in the 3′ noncoding region of the M segment of viral RNA.
12. The chimeric virus of any one of paragraphs 1-9, wherein at least one member of the second set of one or more point mutation(s) is selected from the group consisting of a T261G and an A310G mutation in the influenza virus genome.
13. The chimeric virus of paragraph 12, comprising a third set of one or more mutation(s) that confers replication at a temperature below 35° C.
14. The chimeric virus of paragraph 12, wherein the third set of one or more mutation(s) comprises a third set of one or more point mutation(s) that is distinct from the second set of one or more point mutation(s), and is selected from the group consisting of a T261G and an A310G mutation in the H1N1 influenza virus genome.
15. The chimeric virus of any one of paragraphs 1-13, wherein the one or more CoV2Ag is the Sar-CoV-2 receptor binding domain (RBD).
16. The chimeric virus of any one of paragraph 1-15, selected from the group consisting of CA04-DelNS1-Sars-CoV-2-RBD; HK68-DelNS1-Sars-CoV-2-RBD ; 4801-DelNS1 -Sars-CoV-2-RBD and H1N1(2019)-DelNS1 -Sars -CoV-2-RBD.
17. A pharmaceutical composition comprising an effective amount of the chimeric virus of anyone of paragraphs 1-16, and optionally, LAIVA and/LAIB.
18. The composition of paragraph 17, further comprising an adjuvant.
19. The composition of any one of paragraphs 17 or 18, suitable for nasal administration.
20. A method for increasing an immune response to Sars-CoV-2 in a subject in need thereof, comprising administering the composition of any one of paragraphs 1-13, to the subject.
21. A live attenuated chimeric virus comprising (a) an influenza B virus genome, wherein the influenza B virus genome comprises a deletion of a virulence factor activity (DELNS1-B), and optionally, one or more mutations selected from the group consisting of PA(T210C), NA T(1424C), NP(C182T) and M (A281G) (AM/LAIVB/DelNS1), and (b) an insertion of one or more genes encoding one or more Sars-CoV-2 antigens (CoV2Ag).
22. The attenuated chimeric virus of paragraph 21, wherein the influenza B virus genome is from influenza B (B/HK/8038/2011)(DELNS1-B8038).
23. The attenuated chimeric virus of paragraph 21 or 22, wherein the influenza B virus is not able to replicate in interferon-competent cells.
24. The attenuated chimeric virus any one of paragraphs 21-23, wherein the deletion of virulence factor activity comprises a deletion of at least part of a virulence factor gene.
25. The chimeric virus of any one of paragraphs 21-24, wherein the deletion comprises a complete deletion of the Non-Structural Protein 1 (NS1) gene.
26. The chimeric virus of any one of paragraphs 21-25, comprising a first set of one or more mutation(s), wherein the first set of one or more mutation(s) comprises a first set of one or more point mutation(s) that confer replicative competence, wherein the one or more mutations selected from the group consisting of PA(T210C), NA T(1424C), NP(C182T) and M (A281G) (AM/LAVIB/DelNS1).
27. The chimeric virus of any one of paragraphs 21-26, wherein the virus replicates poorly in MDCK cells at 37° C., when compared to its replication at 33° C. in the MDCK cells.
28. The chimeric virus of any one of paragraphs 21-27, wherein the one or more CoV2Ag is the Sar-CoV-2 receptor binding domain (RBD).
29. The chimeric virus of any one of paragraph 21-28, wherein the chimeric virus is DELNS1-B 8038-Sars-CoV-2-RBD.
30. A pharmaceutical composition comprising an effective amount of the chimeric virus of anyone of paragraphs 21-29.
31. The composition of paragraph 30, further comprising an adjuvant.
32. The composition of any one of paragraphs 30 or 31, suitable for nasal administration.
33. A method for increasing an immune response to Sars-CoV-2 in a subject in need thereof, comprising administering the composition of any one of paragraphs 21-32, to the subject.

### EXAMPLES

### Materials and Methods

### Cells and Viruses

All cell lines were obtained from ATCC. Human cells were maintained in Dulbecco's minimal essential medium (DMEM) supplemented with 10% fetal bovine serum, 100 units/ml penicillin, and 100 μg/ml streptomycin sulfate (Life Technologies). MDCK cells were cultured in Eagle's minimal essential medium (MEM) supplemented with the same amount of serum and antibiotics. CA04-DelNS1 LAIV were constructed and rescued according to the protocols described here and in the previous report (Wang, et al, mBio, 10(5): e02180-19 (2019). Viral gene segments were amplified and cloned into pHW2000 plasmids, resulting in eight pHW2000 plasmids, which were transfected into 293T/MDCK cell mixtures. Rescued virus was amplified in MDCK cells or embryonated chicken eggs. CA04-DelNS1 LAIV was used as backbone for making other DelNS1-SARS-CoV-RBD LAIVs.

Construction of Plasmids

Plasma construction follows the protocol described in Wang, et al, mBio, 10(5): e02180-19 (2019). NS1 deletion plasmid pHW2000-DelNS1 was constructed as described before (Zheng, et al, J. Virol., 89:10273-10285 (2015)). Inverse PCR is performed to delete the NS1 gene using plasmid pHW2000-CA04-NS (influenza A virus). The PCR product was then gel purified, phosphorylated and self-ligated using a standard protocol. Primers for CA04-DeNS1 inverse PCR are 5′-GACATACTTATGAGGATGTC-3′ (SEQ ID NO:29 (CA04-DelNS1-F) and 5′-CTGAAAGCTTGACATGGTGTTG-3′ (SEQ ID NO:30) (CA04-DelNS1-R) (Wang, et al, mBio, 10(5): e02180-19 (2019). A QuikChange II site-directed mutagenesis kit (Agilent) is used to generate point mutations. pHW2000-CA4-DelNS1-SARS-CoV2-RBD was made by cloning of the RBD region of of SARS-CoV-2 into the site of deleted NS1 of CA04-DelNS1. A protease cleavage motif, 2A, was inserted between RBD and the NEP coding region (FIG. 1).

HK68-DelNS1-SARS-CoV-2-RBD was constructed using the backbone of CA04-DelNS1 with hemagglutinin (HA) and neuraminidase (NA) derived from A/HK/01/1968 (H3N2). Similarly, HK4801-DelNS1-SARS-COV-2-RBD AND H1N1(2019)-DelNS1-SARS-COV-2-RBD were constructed in the internal gene backbone of CA04-DelNS 1 with HA and NA derived from strain of A/HK/4801/2014 (H3N2) or A/HK/2019 (H1N1).

Generation and Passage of DeINS1 Viruses.

Eight pHW2000 plasmids containing the DelNS1 segment and the other 7 influenza virus genomic segments, together with an NS1 expression plasmid, were transfected into a 293T/MDCK cell mixture and incubated overnight. The DNA mixture was removed and MEM supplemented with 1 μg/ml N-tosyl-L-phenylalanine chloromethyl ketone (TPCK)-treated trypsin (Sigma) added. Virus supernatant was collected 72 h later and designated passage 0 (P0) virus and subsequently passaged in MDCK cells or embryonated chicken eggs. For CA04-DelNS1 virus, rescued virus was passaged in MDCK cells 10 times at 37° C. and then a further 10 times at 30° C. CA04-DelNS1-SARS-CoV-2-RBD, HK68-DelNS1-SARS-CoV-2-RBD, HK4801-DelNS1-SARS -COV-2-RBD AND H1N1 (2019)-DelNS1-S ARS -COV-2-RBD were rescued and passage similarly as described above.

For all DelNS1-SARS-CoV-2 RNA LAIV viruses, insertion of RBD and deletion of the NS1 gene was confirmed by reverse transcription-PCR (RT-PCR) and sequencing.

RT-PCR

Verification of NS segment and RBD insert in DelNS1-nCoV-RBD vaccine strain

RNA was extracted from DelNS1 vaccine strains, (CA04-DelNS-Sars-CoV-2-RBD, (herein also, CA04-DelNS-nCoV-RBD), HK68-DelNS1-Sars-CoV-2-RBD (herein, also, HK68-DelNS1-nCoV-RBD), 4801-DelNS1-Sars-CoV-2-RBD (herein, also, 4801 -DelNS1 -nCoV-RBD) and H1N1(2019)-DelNS1-Sars-CoV-2-RBD (herein, also, H1N1(2019)-DelNS1-nCoV-RBD), and subsequently, passaged in eggs. RT-PCR with primers specific for the NS segment and RBD of Sars-CoV-2 (herein also, nCoV) were performed and PCR products were analyzed be agarose electrophoresis. Correct size of PCR products, NS and RBD were observed from all DelNS1 vaccine strains.

Verification of expression of Sars-CoV-2 (nCoV) RBD in DelNS1-nCoV-RBD live attenuated virus infected MDCK cells. MDCK Cells were infected with CA04-DelNS-nCoV-RBD, HK68-DelNS1-nCoV-RBD, 4801-DelNS1-nCoV-RBD, or H1N1(2019)-DelNS1-nCoV-RBD at 0.1 MOI, or mock infection for 16 hours. Cell lysates were harvested and analyzed by Western blot using either anti-NP (for viral protein NP) or anti-V5 (for RBD which is tagged with a V5 epitope). As shown in the results, RBD was expressed from all DelNS1 vaccines strains.

Animal Studies

Two groups (six each)of six to eight-week old female DPP4 transgenic mice are anesthetized and then inoculated intranasally with 25 μl PBS containing 5×10⁵ TCID₅₀ of MERS-RBD-DelNS1, DelNS1-MERS-N or control (PBS only), twice, respectively, four weeks apart. Mice were challenged with MERS coronavirus (500 pfu=10 MLD₅₀, or 100 pfu=2 MLD₅₀). Mice were monitored for 14 days for body weight loss and mortality.

### Example 1

### Construction of DeINS1-MERS-RBD and DeINS1-MERS-N LAIV Vaccine Strains

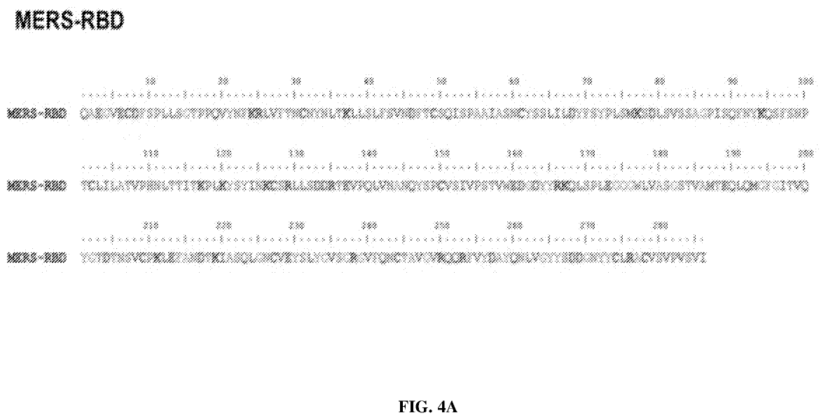

For proof of concept, gene segments containing the RBD and N derived from MERS coronavirus was cloned into NS segment of CA04-DelNS1 LAIV (Wang et al., mBio 10 (5):e12180-19 (2019).) (FIGS. 1A-B). The sequences for the Receptor Binding Domain (RBD) of the MERS coronavirus is shown in FIG. 4A.

### Example 2

### Protection of DPP4 Transgenic Mice with Inoculation of Lethal Challenge of MERS Coronavirus (2 MLD₅₀)

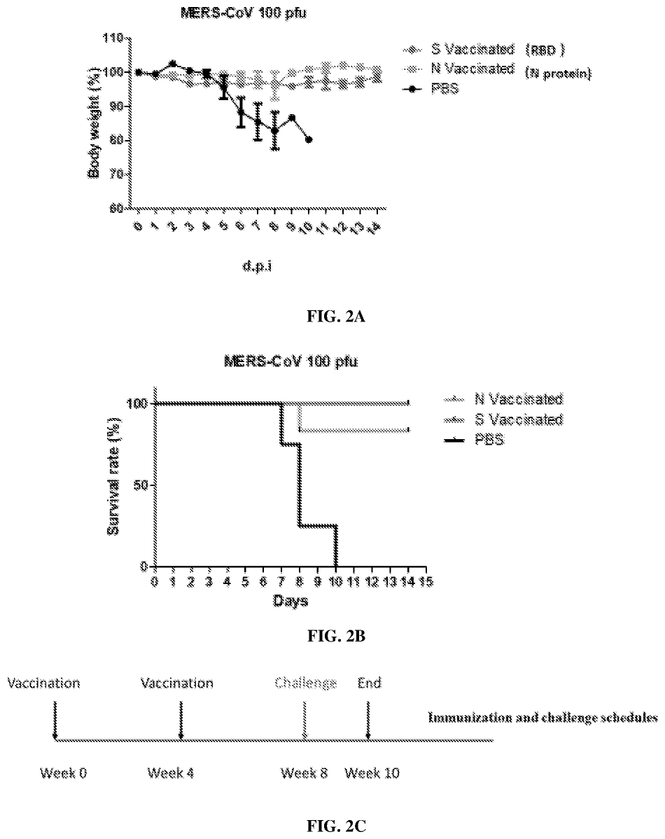

Transgenic mice expressing human DPP4 receptor were prime immunized with DelNS1-MERS-RBD, DelNS1-MERS-N, or control (PBS) twice respectively, four weeks apart. Immunized mice were then challenged with lethal dose of MERS coronavirus (100 pfu=2 MLD₅₀). Mice were monitor for 14 days for body weight loss and mortality. The data is shown in FIGS. 2A and 2B

### Example 3

### Protection of DPP4 Transgenic Mice with Inoculation of Lethal Challenge of MERS Coronavirus (10 MLD₅₀)

Transgenic mice expressing human DPP4 receptor were prime immunized with DelNS1-MERS-RBD LAIV, DelNS1-MERS-N LAIV, or DelNS1 LAIV twice respectively, four weeks apart Immunized mice were then challenged with lethal dose of MERS coronavirus (500 pfu=10 MLD₅₀). Mice were monitored for 14 days for body weight loss and mortality.

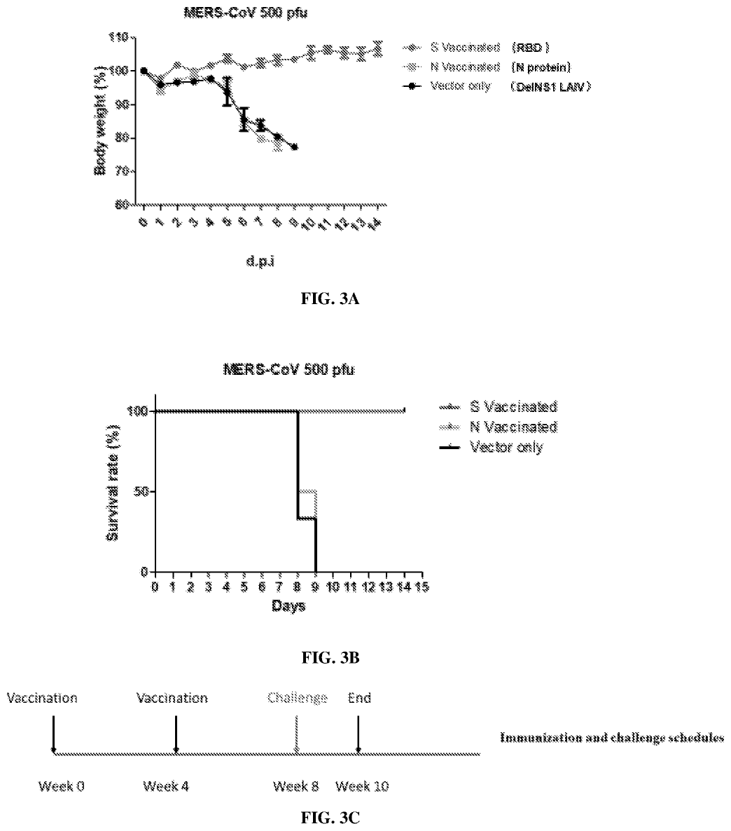

The data is shown in FIGS. 3A and 3B

### Example 4

### Cloning of 2019 Novel Coronavirus (Sars-CoV-2) into DeINS1 LAIV Vector

The sequences for the Receptor Binding Domain of the Sars-CoV-2 is shown in FIG. 4B.

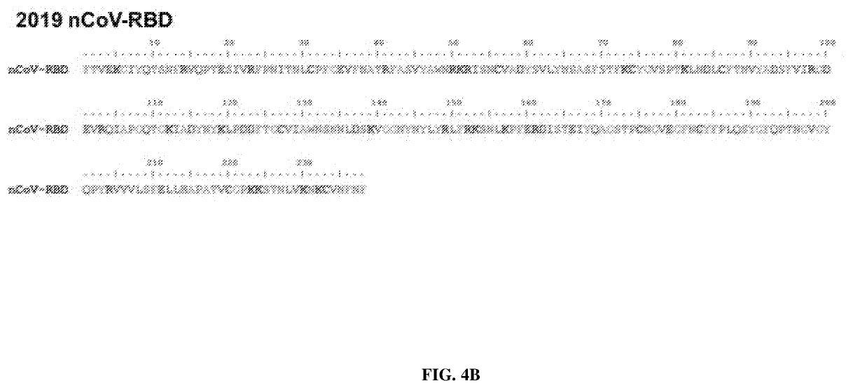

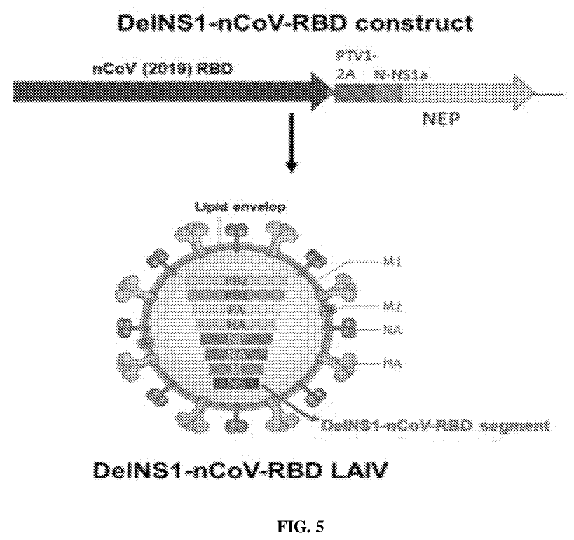

The gene segment containing the RBD from Sars-CoV-2 was cloned into NS segment of CA04-DelNS1 LAIV (Wang et al., mBio 10 (5):e12180-19 (2019)) as depicted in FIG. 5. Verification of NS segment and RBD insert in DelNS1-Sars-CoV-2-RBD vaccine strain is shown in FIG. 6. RNAs were extracted from DelNS1 vaccine strains, CA04-DelNS-Sars-CoV-2-RBD, HK68-DelNS1-Sars-CoV-2-RBD, 4801 -DelNS1-Sars-CoV-2-RBD and H1N1(2019)-DelNS1-Sars-CoV-2-RBD, after passage in eggs. RT-PCR with primers specific for the NS segment and RBD of Sars-CoV-2 were performed and PCR products were analyzed be agarose electrophoresis. Correct size of PCR products, NS an RBD were observed from all DelNS1 vaccine strains.

### Example 5

### Expression of Sars-CoV-2 RBD in DeINS1-Sars-CoV-2-RBD Live Attenuated Virus Infected MDCK Cells

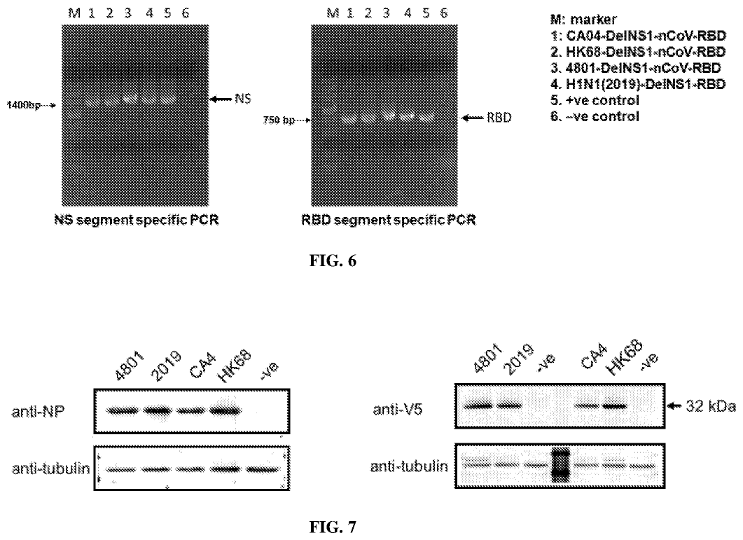

MDCK Cells were infected with CA04-DelNS-Sars-CoV-2-RBD, HK68-DelNS1-Sars-CoV-2-RBD, 4801 -DelNS1 -Sars-CoV-2-RBD, or H1N1(2019)-DelNS1-Sars-CoV-2-RBD at 0.1 MOI, or mock infection for 16 hours. Cell lysates were harvested and analyzed by Western blot using either anti-NP (for viral protein NP) or anti-V5 (for RBD which is tagged with a V5 epitope). It is shown that RBD is expressed from all DelNS1 vaccines strains (FIG. 7).

### Example 6

### Protection of ACE2 Transgenic Mice from Disease Caused by SARS-CoV-2 Infection

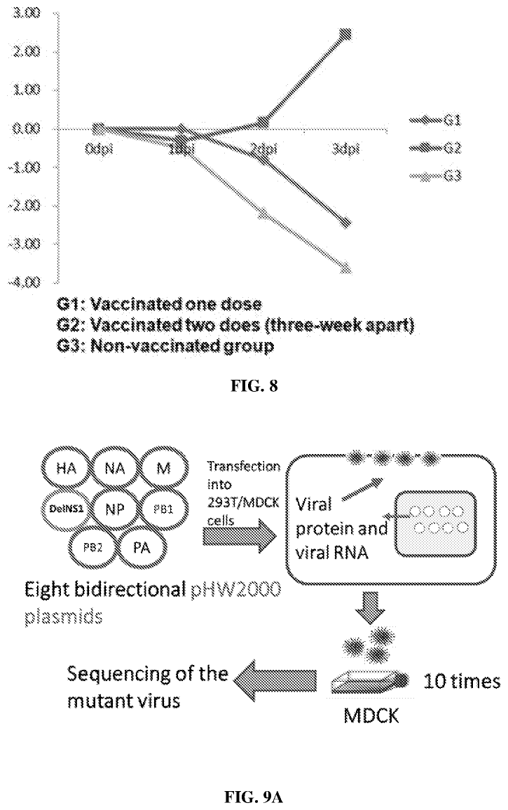

ACE2 transgenic mice were inoculated with CA04-DelNS-Sars-CoV-2-RBD LAIV once or twice (in three-week apart). Three weeks after the last vaccination, mice were challenged with 1×10 ⁵ TCID₅₀ of SARS-CoV-2 or PBS (control). Mice were observed for body weight change after virus challenge (FIG. 8). Mice immunized with CA04-DelNS-Sars-CoV-2-RBD LAIV show less body weight loss (one dose) or no body weight loss and gain body weight after three days post infection (two doses).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different moieties does not indicate that the listed moieties are obvious one to the other, nor is it an admission of equivalence or obviousness.

1. A live attenuated chimeric virus comprising (a) an influenza A virus genome, wherein the influenza A virus genome comprises a deletion of a virulence factor activity, and optionally, a first set of one or more mutation(s) that confers replication at 37° C. in the absence of the virulence factor activity; and a second set of one or more mutation(s) that confers replication at a temperature below 35° C., or an influenza B virus genome, wherein the influenza B virus genome comprises a deletion of a virulence factor activity (DELNS1-B), and optionally, one or more mutations selected from the group consisting of PA(T210C), NA T(1424C), NP(C182T) and M (A281G) (AM/LAIVB/DelNS1), and (b) an insertion of one or more genes encoding one or more Sars-CoV-2 antigens (CoV2Ag) or one or more genes encoding one or more hemagglutinin (H) or neruaminidase (N) subtypes selected from the group consisting of H1, H2, H3, H5, H6, H7, H9, H10, N1, N2, N6-N9.

2. The attenuated chimeric virus of claim 1, wherein the influenza virus genome is from: (a) an influenza virus A subtype H1N1 or H3N2, or (b) influenza B (B/HK/8038/2011)(DELNS1-B8038).

3. The attenuated chimeric virus of claim 1, wherein (a) the influenza virus genome is from an influenza virus A subtype H1N1 or H3N2 strain selected from the group consisting of CA04 (A/California/04/2009); HK68 (strain A/Hong Kong/1/68), 4801 (H3N2 A/HK/4801/2014), H1N1(2019); A/WSN/33 and A/PR/8/34; or (b) wherein the influenza B virus is not able to replicate in interferon-competent cells.

4. The attenuated chimeric virus of claim 1, wherein the deletion of virulence factor activity comprises a deletion of at least part of a virulence factor gene.

5. The chimeric virus of claim 1, wherein the deletion comprises a deletion of at least part of Non-Structural Protein 1 (NS1) gene extending beyond nucleotides 57 to 528 of an NS1 segment of the mutated virus.

6. The chimeric virus of claim 1, comprising a first set of one or more mutation(s), wherein the first set of one or more mutation(s) comprises a first set of one or more point mutation(s) that confer replicative competence, optionally wherein the chimeric virus is a LAIVB and wherein the one or more mutations are selected from the group consisting of PA(T210C), NA T(1424C), NP(C182T) and M (A281G) (AM/LAVIB/DelNS 1).

7. The chimeric virus of claim 1, wherein the first set of one or more point mutation(s) lies outside of an M region of the mutated influenza virus.

8. The chimeric virus of claim 3, wherein the influenza virus genome is from the A/California/04/2009 influenza strain, and at least one of the first set of one or more point mutation(s) is a G346A mutation in the viral genome; the chimeric virus is B8038-DELNS1-Sars-CoV-2-RBD or wherein the one or more CoV2Ag is the Sar-CoV-2 receptor binding domain (RBD).

9. The chimeric virus of claim 1, wherein the virus replicates poorly in MDCK cells at 37° C., when compared to its replication at 33° C. in the MDCK cells and/or the second set of one or more mutation(s) comprises a second set of one or more point mutation(s).

10. (canceled)

11. The chimeric virus of claim 3, wherein the first set of one or more mutation(s) comprises an A14U substitution in the 3′ noncoding region of the M segment of viral RNA and the chimeric virus is a DELNS1-A/B-Sars-CoV-2-CoV2Ag.

12. The chimeric virus of claim 1, wherein at least one member of the second set of one or more point mutation(s) is selected from the group consisting of a T261G and an A310G mutation in the influenza virus genome and the chimeric virus is a DELNS1-A/B-Sars-CoV-2-CoV2Ag, and/or comprising a third set of one or more mutation(s) that confers replication at a temperature below 35° C.

13. (canceled)

14. The chimeric virus of claim 12, wherein the third set of one or more mutation(s) comprises a third set of one or more point mutation(s) that is distinct from the second set of one or more point mutation(s), and is selected from the group consisting of a T261G and an A310G mutation in the H1N1 influenza virus genome and the chimeric virus is a DELNS1-A/B-Sars-CoV-2-CoV2Ag.

15. The chimeric virus of claim 1, wherein the antigen is not full length spike protein of Sars-CoV-2, and optionally, wherein the one or more CoV2Ag is the Sar-CoV-2 receptor binding domain (RBD).

16. The chimeric virus of claim 1, selected from the group consisting of CA04-DeINS1-Sars-CoV-2-RBD; HK68-DelNS1-Sars-CoV-2-RBD ; 4801-DeINS1-Sars-CoV-2-RBD and H1N1(2019)-DelNS1-Sars-CoV-2-RBD.

17. A pharmaceutical composition comprising an effective amount of the chimeric virus of claim 1.

18. The composition of claim 17, further comprising an adjuvant.

19. The composition of claim 17: (a) in a form suitable for nasal administration, (b) further comprising a LAIVA, LAIVB, CA4-DelNS1 and/or DeINS1-B8038HK.

20. (canceled)

21. (canceled)

22. A method for increasing an immune response to Sars-CoV-2 in a subject in need thereof, comprising administering to the subject:
(a) the chimeric virus of claim 1, or
(b) a pharmaceutical composition comprising the composition of claim 1.

23. The method of claim 22, wherein the virus is administered intranasally or intramuscularly.

24. (canceled)

25. A method for increasing an immune response to Sars-CoV-2 and influenza in a subject in need thereof, comprising administering the composition of claim 19 to the subject.
